(19) United States
(12) Patent Application Publication
(10) Pub. No.: US 2026/0101991 A1
(43) Pub. Date: Apr. 16, 2026

(54) APPARATUS, SYSTEM AND METHOD FOR DETECTING BIOMARKERS

(71) Applicant: Colgate-Palmolive Company, New York, NY (US)

(72) Inventors: Indrani BANERJEE, Basking Ridge, NJ (US); Donghui WU, Bridgewater, NJ (US); Bryan LEE, Somerset, NJ (US)

(73) Assignee: Colgate-Palmolive Company, New York, NY (US)

(21) Appl. No.: 19/114,622

(22) PCT Filed: Sep. 13, 2023
(86) PCT No.: PCT/US2023/074079

§ 371 Date: Mar. 24, 2025

Related U.S. Application Data
(60) Provisional application No. 63/417,530, filed on Oct. 19, 2022.

(51) Int. Cl. A46B 15/00; A46B 5/00; A46B 13/02; A61B 10/00; A61C 17/22; G01N 21/31; G01N 33/487

(52) U.S. Cl. CPC A46B 15/0048; A46B 5/0095; A46B 13/023; A46B 15/0038; A61B 10/0051; A61C 17/222; A61C 17/227; G01N 21/31; G01N 33/487; G01N 2201/062

(57) ABSTRACT

An oral care implement, oral care system, and/or method for detecting the presence or absence of hemoglobin/blood in a user's oral cavity. The oral care implement may include a cavity for receiving a saliva sample from a user and a closure member that can close the cavity for purposes discussed herein. The oral care system may include a handle, a refill head, and a collection cup, with the refill head and collection cup being detachably coupled to the handle. A sample of the user's saliva may be introduced into the collection cup. The handle may include a sensor that can obtain information related to the presence or absence of hemoglobin/blood in the oral cavity or in a sample of the user's saliva. The sensor may be operably coupled to a processor that can determine whether hemoglobin is present in the sample based on the information obtained by the sensor.

### CROSS-REFERENCE TO RELATED APPLICATION

This application claims the benefit of priority from U.S. Provisional Application No. 63/417,530, filed Oct. 19, 2022, the contents of which are hereby incorporated herein by reference in their entirety.

### BACKGROUND

There are several different causes for gum and other oral cavity bleeding. Some of them are mechanical issues, such as excessive brushing force, using a toothbrush with stiff bristles, incorrect brushing or flossing mechanics, or improperly fitted dentures. Gum disease can also cause bleeding due to inflammation of gum tissues. Other important causes can be systemic, such as medications (e.g. blood thinners), pregnancy (hormonal changes), diabetes, vitamin deficiency (e.g. C or K), Leukemia, etc. Although the American Dental Association recommends that people with gum bleeding see a dentist or physician, a majority of the population do not feel pain when they have bleeding gums and therefore ignore this recommendation. Therefore, there is a need to provide consumers with an apparatus, system, and/or method for detecting blood in the oral cavity.

### BRIEF SUMMARY

The present invention may be directed to an oral care implement, oral care system, and/or method for detecting the presence or absence of hemoglobin/blood in a user's oral cavity. The oral care implement may include a cavity for receiving a saliva sample from a user and a closure member that can close the cavity for purposes discussed herein. The oral care system may include a handle, a refill head, and a collection cup, with the refill head and collection cup being detachably coupled to the handle. A sample of the user's saliva may be introduced into the collection cup. The handle may include a sensor that can obtain information related to the presence or absence of hemoglobin/blood in the oral cavity or in a sample of the user's saliva. The sensor may be operably coupled to a processor that can determine whether hemoglobin is present in the sample based on the information obtained by the sensor.

In one aspect, the invention may be an oral care implement comprising: a body comprising a cavity configured to receive a sample of a user's saliva, the cavity comprising a first opening through which the user's saliva can be introduced into the cavity; a first closure member alterable between: (1) a closed state whereby the first closure member closes the first opening of the cavity and prevents the user's saliva from entering into or exiting the cavity through the first opening; and (2) an open state whereby the first opening provides a first passageway into the cavity through which the user's saliva may be introduced into or removed from the cavity; and at least one sensor configured to emit light into the cavity and receive reflected portions of the emitted light to obtain data useful for determining whether hemoglobin is present in the user's saliva located in the cavity.

In another aspect, the invention may be an oral care system comprising: a handle comprising a gripping portion and a stem portion, the handle comprising at least one sensor; a refill head comprising a sleeve portion and a head portion having tooth cleaning elements extending therefrom, the refill head configured to be detachably coupled to the handle; a collection cup comprising a collection cavity, the collection cup configured to be detachably coupled to the handle; and wherein when the collection cup is coupled to the handle and a sample of a user's saliva is located in the collection cavity, the at least one sensor is configured to obtain data useful for determining whether hemoglobin is present in the sample of the user's saliva located in the collection cavity.

In another aspect, the invention may be a method for detecting blood in an oral cavity, the method comprising: performing a toothbrushing session with a toothbrush comprising a handle and a refill head that is detachably coupled to the handle, the refill head comprising tooth cleaning elements that engage teeth and gums of an oral cavity of a user during the toothbrushing session; upon completion of the toothbrushing session, detaching the refill head from the handle; attaching a collection cup to the handle of the toothbrush; prior to rinsing the user's mouth, introducing a sample of saliva from the user's mouth into the collection cavity of the collection cup; emitting into the collection cavity, via a sensor located within the handle of the toothbrush, first light at a first wavelength and second light at a second wavelength; receiving, via the sensor of the toothbrush, reflected portions of the first light and the second light; and transmitting, from the sensor to a processor, a first signal indicative of a first intensity of the reflected portion of the first light and a second signal indicative of a second intensity of the reflected portion of the second light; and calculating, via the processor, a ratio of the first intensity to the second intensity to determine whether hemoglobin is present in the sample.

In yet another aspect, the invention may be an oral care system comprising: a handle comprising a gripping portion and a stem portion, the handle comprising at least one sensor; a collection cup comprising a top end, a bottom end, a collection cavity comprising a floor, and a channel that is distinct from the collection cavity; wherein the collection cup is configured to be detachably coupled to the handle by inserting at least a distal portion of the stem portion of the handle into the channel of the collection cup so that the at least one sensor of the handle is aligned with the collection cavity; and wherein when the collection cup is coupled to the handle and a sample of a user's saliva is located in the collection cavity, the at least one sensor is configured to obtain data useful for determining whether hemoglobin is present in the sample of the user's saliva located in the collection cavity.

In a further aspect, the invention may be an oral care system comprising: a handle comprising a gripping portion and a stem portion, the handle comprising at least one sensor; a collection cup comprising a collection cavity; wherein the collection cup is configured to be detachably coupled to the handle so that the at least one sensor of the handle is adjacent to or aligned with the collection cavity; and wherein when the collection cup is coupled to the handle and a sample of a user's saliva is located in the collection cavity, the at least one sensor is configured to obtain data useful for determining whether hemoglobin is present in the sample of the user's saliva located in the collection cavity.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a front perspective view of an oral care implement in accordance with an embodiment of the present invention;

FIG. 2 is a rear perspective view of the oral care implement of FIG. 1;

FIG. 3 is an exploded front perspective view of the oral care implement of FIG. 1 illustrating a refill head thereof separated from a handle thereof;

FIG. 4 is a cross-sectional view taken along line IV-IV of FIG. 1;

FIG. 4A is a close-up view area IVA of FIG. 4, whereby closure members are in an open state;

FIG. 5 is a schematic illustration of a sensor of the oral care implement of FIG. 1;

FIGS. 6A and 6B are schematic illustrations of the sensor of FIG. 5 transmitting light into a saliva sample and receiving reflected light in accordance with embodiments of the present invention;

FIG. 7 is a close-up perspective view of a head portion of the oral care implement of FIG. 1 illustrating a cover member in a closed state;

FIG. 8 is a close-up perspective view of a head portion of the oral care implement of FIG. 1 illustrating a cover member in an open state;

FIG. 9 is a schematic view illustrating a user depositing a saliva sample into a cavity of the oral care implement of FIG. 1;

FIG. 10 is a schematic view illustrating the cavity of the oral care implement being rinsed;

FIG. 11 illustrates a system for detecting blood in an oral cavity that includes an oral care implement and a portable electronic device that are in operable communication with one another;

FIG. 12 is an electrical block diagram of the electronic components of the oral care implement and the portable electronic device of the system of FIG. 11;

FIG. 13 is a perspective view of an oral care system in accordance with an embodiment of the present invention, the oral care system including a handle, a refill head, and a collection cup;

FIG. 14 is a perspective view of the handle and the refill head of the oral care system of FIG. 13 in an attached state;

FIG. 15 is a top perspective view of the collection cup of the oral care system of FIG. 13;

FIG. 16 is a top view of the collection cup of FIG. 13;

FIG. 17 is a cross-sectional view taken along line XVII-XVII of FIG. 16;

FIG. 18A is a cross-sectional view taken along line XVIII-XVIII of FIG. 16 in accordance with a first embodiment of the present invention;

FIG. 18B is a cross-sectional view taken along line XVIII-XVIII of FIG. 16 in accordance with a second embodiment of the present invention;

FIG. 19 is a rear perspective view of the handle and the collection cup of the oral care system of FIG. 13 in an attached state;

FIG. 20 is a front view of the handle and the collection cup of the oral care system of FIG. 13 in an attached state;

FIG. 21 is a cross-sectional area taken along line XXI-XXI of FIG. 20, whereby a sample of a user's saliva is located in a collection cavity of the collection cup;

FIG. 22 is a front perspective view of a head of an oral care implement in accordance with an embodiment of the present invention;

FIG. 23 is a cross-sectional view taken along line XXIII-XXIII of FIG. 22 in accordance with one embodiment of the present invention;

FIG. 24 is a cross-sectional view taken along line XXIII-XXIII of FIG. 22 in accordance with another embodiment of the present invention;

FIG. 25 is a front perspective view of a head of an oral care implement in accordance with an embodiment of the present invention;

FIG. 26 is a cross-sectional view taken along line XXVI-XXVI of FIG. 25 in accordance with one embodiment of the present invention; and

FIG. 27 is a cross-sectional view taken along line XXVI-XXVI of FIG. 25 in accordance with another embodiment of the present invention.

### DETAILED DESCRIPTION

The following description of the preferred embodiment(s) is merely exemplary in nature and is in no way intended to limit the invention, its application, or uses.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivatives thereof (e.g., “horizontally.” “downwardly.” “upwardly.” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed.” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

Features of the present invention may be implemented in software, hardware, firmware, or combinations thereof. The computer or software programs described herein are not limited to any particular embodiment, and may be implemented in an operating system, application program, foreground or background processes, driver, or any combination thereof. The computer programs may be executed on a single computer or server processor or multiple computer or server processors.

Processors described herein may be any central processing unit (CPU), microprocessor, micro-controller, computational, or programmable device or circuit configured for executing computer program instructions (e.g. code). Various processors may be embodied in computer and/or server hardware of any suitable type (e.g. desktop, laptop, notebook, tablets, cellular phones, etc.) and may include all the usual ancillary components necessary to form a functional data processing device including without limitation a bus, software and data storage such as volatile and non-volatile memory, input/output devices, graphical user interfaces (GUIs), removable data storage, and wired and/or wireless communication interface devices including Wi-Fi, Bluetooth, LAN, etc.

Computer-executable instructions or programs (e.g. software or code) and data described herein may be programmed into and tangibly embodied in a non-transitory computer-readable medium that is accessible to and retrievable by a respective processor as described herein which configures and directs the processor to perform the desired functions and processes by executing the instructions encoded in the medium. It should be noted that non-transitory “computer-readable medium” as described herein may include, without limitation, any suitable volatile or non-volatile memory including random access memory (RAM) and various types thereof, read-only memory (ROM) and various types thereof, USB flash memory, and magnetic or optical data storage devices (e.g. internal/external hard disks, floppy discs, magnetic tape CD-ROM, DVD-ROM, optical disk, ZIP™ drive, Blu-ray disk, and others), which may be written to and/or read by a processor operably connected to the medium.

In certain embodiments, the present invention or components thereof may be embodied in the form of computer-implemented processes and apparatuses such as processor-based data processing and communication systems or computer systems for practicing those processes. The present invention may also be embodied in the form of software or computer program code embodied in a non-transitory computer-readable storage medium, which when loaded into and executed by the data processing and communications systems or computer systems, the computer program code segments configure the processor to create specific logic circuits configured for implementing the processes.

The invention described herein relates to an apparatus (i.e., oral care implement such as a toothbrush), system, and method of detecting one or more biomarkers in an oral cavity. In one embodiment, the biomarkers may be blood or hemoglobin. Thus, the apparatus, system, and/or method may detect the presence of blood using sensors that measure the hemoglobin level in saliva/toothpaste slurry. As used herein, the term saliva includes pure saliva as well as saliva mixed with toothpaste which may be referred to herein as a toothpaste slurry. Toothpaste slurry is the fluid that is formed in a user's mouth during toothbrushing, and is encompassed in the definition of the term saliva as used herein. In one aspect, the detection of blood in the oral cavity may occur during toothbrushing, so the sensors may be described herein as being located on a toothbrush. In another aspect, the detection of blood in the oral cavity may be accomplished by testing a sample of a user's saliva after completion of a toothbrushing session, or at any time desired by the user. The sensor used for detecting the presence of hemoglobin (which is indicative of blood) may include a light transmitter and a light receiver. The light transmitter may emit visible and infrared light and the intensity of the reflected light (i.e., the light reflected back from the saliva sample/toothpaste slurry) is received by the light receiver. Because hemoglobin has a strong red color, it absorbs green light while reflecting the majority of the red and infrared light back. Therefore, using a ratio of reflected light intensity (red/green and/or infrared/green) and applying that data to a processing algorithm, the hemoglobin can be quantified. The information obtained can either be stored on a memory device in the toothbrush or automatically transferred to a mobile phone (or other portable electronic device) app (software application), or both. In either case, the information can be provided to a user (either as logs of all information or as an indicator that blood was present in the toothpaste slurry) so that a user can be informed about gum bleeding.

Additional information about the hemoglobin/blood detection techniques relevant to the disclosure set forth herein may be found in United States Patent Publication No. 2022/0257968 and International Patent Publication No. WO2022/098552, the entireties of which are incorporated herein by reference.

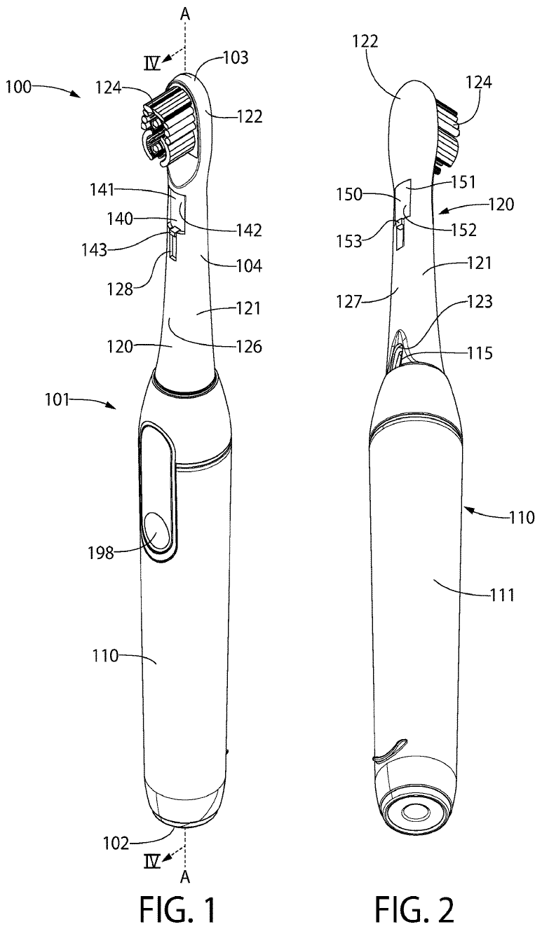

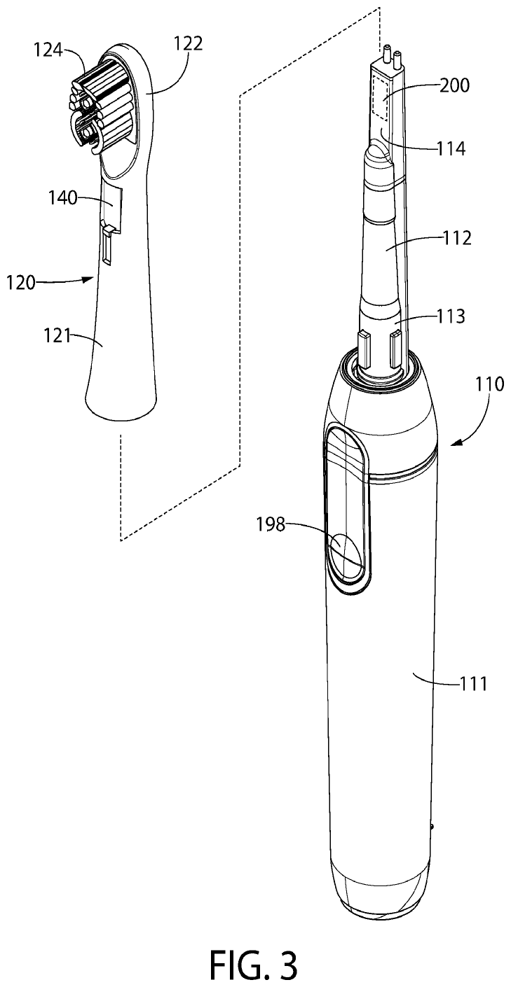

Referring to FIGS. 1-3, an oral care implement 100 is illustrated in accordance with an embodiment of the present invention. In the exemplified embodiment, the oral care implement 100 comprises a body 101 which extends from a proximal end 102 to a distal end 103 along a longitudinal axis A-A. The body 101 may comprise a handle portion 110 and a refill head 120 that is detachably coupled to the handle portion 110. In other embodiments, the body 101 may be an integral structure which comprises a handle portion and a head portion that are integrally formed and attached.

The handle portion 110 of the oral care implement 100 comprises a gripping portion 111 that is generally configured for gripping and handling by a user and a stem portion 112 that facilitates the attachment of the refill head 120 to the handle portion 110. The refill head 120 comprises a sleeve portion 121 and a head portion 122. The sleeve portion 121 is sized and configured to fit over the stem portion 112 of the handle portion 110 for coupling the refill head 120 to the body 110. Thus, the stem portion 112 of the handle portion 110 nests within the sleeve portion 121 (and possibly also the head portion 122) of the refill head 120 when the refill head 120 is coupled to the handle portion 110. The refill head 120 may be coupled to the handle portion 110 with a friction/interference fit or via mechanical interaction, such as the refill head 120 having a protuberance or recess that matches with a recess or protuberance on the handle portion 110. As best shown in FIG. 2, the handle portion 110 may comprise a protuberance 115 on a lower portion of the stem portion 112 which nests within a recess or aperture 123 formed into the refill head 120, and more specifically the sleeve portion 121 thereof. In other embodiments, different techniques for coupling the refill head 120 to the handle portion 110 of the oral care implement 100 could be used in accordance with the invention described herein (i.e., magnetic, mechanical, interference, screw threads, protuberance/detent, or the like). The refill head 120 and the handle portion 110 are illustrated generically and the invention is not to be limited by the shape, size, and/or geometry of these components.

The refill head 120 also comprises cleaning elements 124 that extend from the head portion 122. The cleaning elements 124 may comprise bristles, elastomeric fingers, lamella, rubber elements, combinations thereof, or the like. Specifically, the cleaning elements 124 may be any feature or structure that is known to be used for cleaning of the teeth, gums, and other oral cavity surfaces. The pattern, configuration, material, shape, rigidity, or the like of the cleaning elements is not to be limiting of the invention. The tooth cleaning elements 124 of the present invention can be connected to the head portion 122 of the refill head 120 in any manner known in the art. For example, staples/anchors, in-mold tufting (IMT), anchor free tufting (AFT), PTt anchorless tufting, or the like could be used to mount the cleaning elements/tooth engaging elements 124 to the head portion 122 of the refill head 120. Tooth cleaning elements 124 formed from an elastomeric material may be injection molded onto the head portion 122 of the refill head 120.

In certain embodiments, the exact structure, pattern, orientation, and material of the tooth cleaning elements 124 are not to be limiting of the present invention. Thus, the term “tooth cleaning elements” may be used herein in a generic sense to refer to any structure that can be used to clean, polish or wipe the teeth and/or soft oral tissue (e.g. tongue, check, gums, etc.) through relative surface contact. Common examples of “tooth cleaning elements” include, without limitation, bristle tufts, filament bristles, fiber bristles, nylon bristles, spiral bristles, rubber bristles, elastomeric protrusions, flexible polymer protrusions, combinations thereof, and/or structures containing such materials or combinations. Suitable elastomeric materials include any biocompatible resilient material suitable for uses in an oral hygiene apparatus. To provide optimum comfort as well as cleaning benefits, the elastomeric material of the tooth or soft tissue engaging elements has a hardness property in the range of A8 to A25 Shore hardness. One suitable elastomeric material is styrene-ethylene/butylene-styrene block copolymer (SEBS) manufactured by GLS Corporation. Nevertheless, SEBS material from other manufacturers or other materials within and outside the noted hardness range could be used.

Although in the exemplified embodiment the oral care implement 100 is one which includes a handle portion 110 and a refill head 120 that are detachably coupled together, in other embodiments the oral care implement 100 may be a unitary component comprising a handle and a head that are fixedly coupled together, such as with most traditional manual toothbrushes in the market. Having a detachable refill head 120 may be desirable because it can prolong the use of the oral care implement 100 by enabling a user to replace the refill head 120 and hence also the bristles 124 as they become worn while reusing the handle portion 110 which may include expensive circuitry and electronic components as described further herein below. However, it is possible to utilize the techniques and components described herein on a more conventional manual toothbrush that does not include a replaceable refill head. Thus, the invention is not limited to one which requires the toothbrush to include a refill head in all embodiments unless specifically claimed as such. As noted herein, the body 101 refers to the handle portion 110 and the head portion 122 regardless of whether they are formed as an integral structure or as part of two distinct components which are configured to be detachably coupled together. The oral care implement 100 may further comprise a neck portion 104 located between the gripping portion 111 of the handle portion 110 and the head portion 122. The neck portion 104 may be formed by the sleeve portion 121 of the refill head 120.

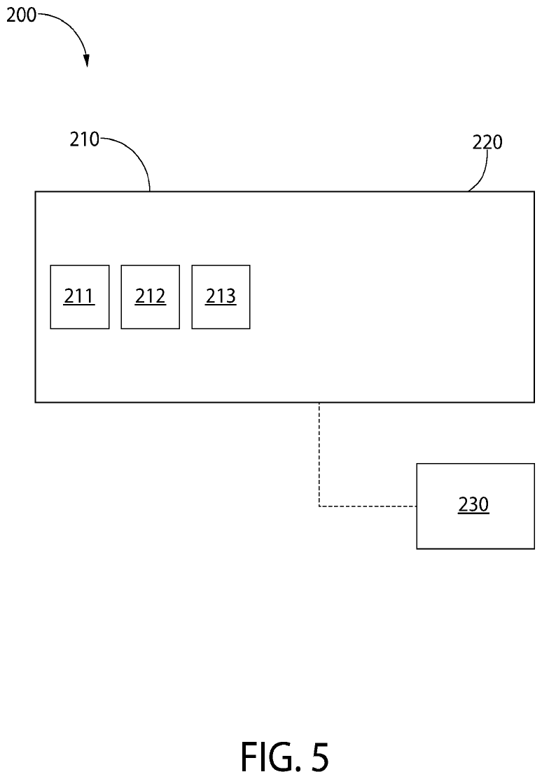

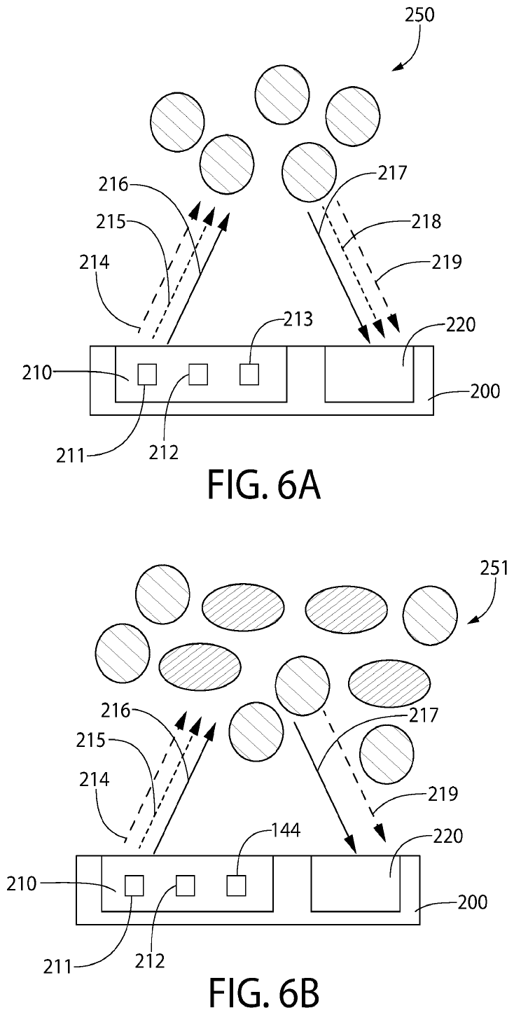

Referring to FIGS. 1-4, the oral care implement 100 comprises at least one sensor 200 that is configured to obtain data useful for determining whether hemoglobin (and therefore, blood) is present in a sample of the user's saliva, obtained as described herein below. In the exemplified embodiment, the at least one sensor 200 is located along, and more specifically within, the stem 112 of the handle portion 110. The stem 112 is elongated along the longitudinal axis A-A and comprises a proximal portion 113 that is adjacent to the gripping portion 111 and a distal portion 114 that is located furthest from the gripping portion 111. The at least one sensor 200 may be located along or within the distal portion 114 of the stem 112. However, the invention is not to be so limited in all embodiments and the location of the sensor 200 may be modified and may be dependent upon a location of a cavity within which a sample of the user's saliva may be introduced. That is, in some embodiments the sensor 200 may be located adjacent to or in alignment with the cavity, which will be described further below. Additional details regarding the sensor will also be provided below with reference to FIGS. 5, 6A and 6B.

Briefly, the oral care implement 100 comprises an electronic circuit which comprises the sensor for acquiring and/or generating signals related to detecting the presence of hemoglobin (and hence also blood) in a sample of a user's saliva (or a toothpaste slurry). The oral care implement 100 (and related system described below) is able to detect and measure hemoglobin/blood using a few wavelengths (two to three or more) in visible and/or infrared regions. Thus, no reagents, assays, wet chemistry preparation, and/or specialized equipment is needed to detect the hemoglobin/blood using the techniques described herein.

Components of the electronic circuit may be located within the handle portion 110 of the oral care implement 100, which is a non-replaceable part of the toothbrush. In that regard, the handle portion 110 (which may include the gripping portion 111 and the stem portion 112) of the oral care implement 100 may comprise a cavity or otherwise hollow region 116 within which the components of the electronic circuit, such as the sensor, are located. Because the electronic circuit is located within the handle portion 110 and not the refill head 120, as the cleaning elements 124 on the refill head 120 become worn, a new refill head 120 can be attached to the handle portion 110 to prolong the usable life of the oral care implement 100. This is one reason that forming the oral care implement 100 so that it includes a handle portion 110 and a detachable refill head 120 may be desirable.

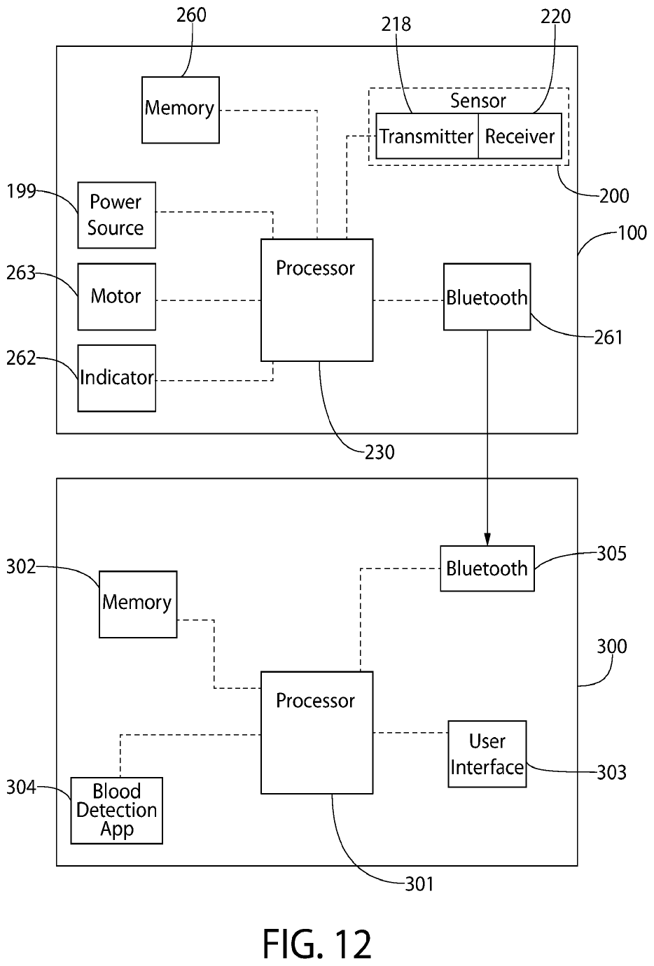

The oral care implement 100 also comprises a power source 199 which is operably coupled to the sensor 200 to provide power to the sensor 200. The power source 199 may form part of the electronic circuit mentioned above. The power source 199 may be a battery located within the gripping portion 111 of the handle portion 110, although the specific location of the power source 199 may not be limiting of the invention in all embodiments. That is, the power source 199 may be located along or within the stem portion 112 in some embodiments. Additional details regarding various electronic components that may be housed within the oral care implement 100 will be discussed below with reference to FIG. 12.

The oral care implement 100 may comprise a power button 198 located along the exterior of the gripping portion 111 of the handle portion 110. The power button 109 may be a depressible button configured to power the oral care implement 100 on and off, which may include powering the sensor 200 on and off and powering a vibrational unit on and off which may cause the tooth cleaning elements 124 to vibrate to perform an effective cleaning of the teeth and gums. That is, the oral care implement 100 may be a powered toothbrush in some embodiments. Of course, the oral care implement 100 may be a manual toothbrush in other embodiments and as such may not include any vibrational elements. The power button 109 may alternatively be a slidable switch, a conductive switch, or other types of switches commonly used for toothbrush or other oral care implement operation.

The body 101 of the oral care implement 100 may comprise a cavity 130 which is configured to receive a sample of a user's saliva (which may comprise a toothpaste slurry, as discussed herein). In the exemplified embodiment, the cavity 130 is located along the refill head 120 of the body 101. That is, the cavity 130 extends from a first opening 131 in a front surface 126 of the refill head 120 to a second opening 132 in a rear surface 127 of the refill head 120. However, the exact location of the cavity 130 may be modified. For example, the cavity 130 may be located along the gripping portion 111 of the handle portion 110 in some embodiments. In such embodiments, the sensor 200 may also be relocated so as to be positioned adjacent to or in alignment with the cavity. Moreover, in embodiments whereby the body 101 is a unitary/monolithic structure such that the head is affixed to the handle portion 110 rather than being formed as part of a replaceable refill head, the cavity 130 may be formed in the neck 104 of such embodiment. Thus, variations are possible which may fall within the scope of the invention.

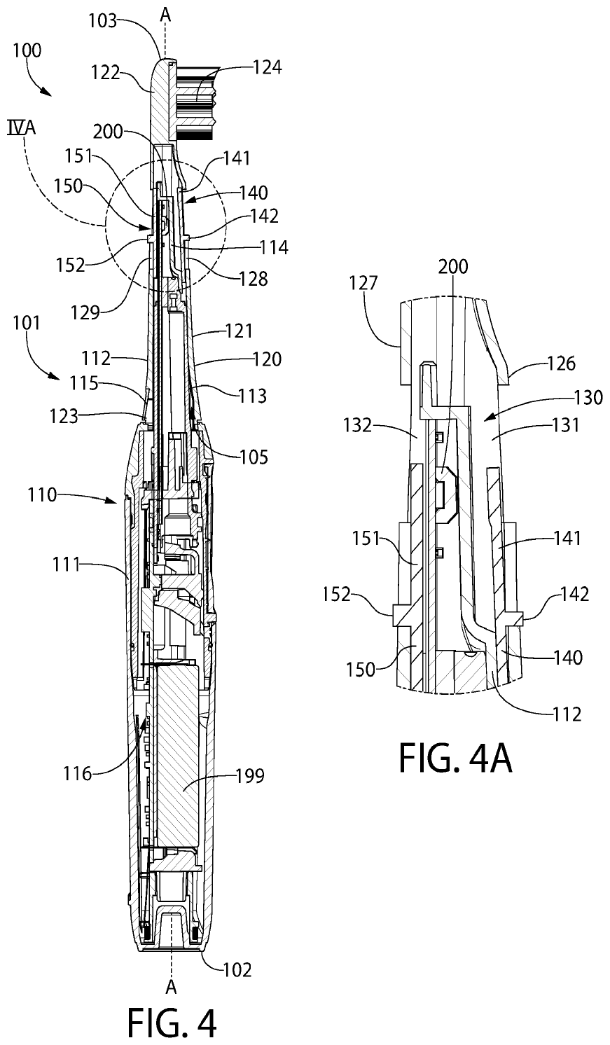

The oral care implement 100 may comprise a first closure member 140 that is alterable between: (1) a closed state whereby the first closure member 140 closes the first opening 131 of the cavity 130; and (2) an open state whereby the first closure member 140 does not completely close the first opening 131 of the cavity 130 so that a passageway exists into the cavity 130 through the first opening 131. In the exemplified embodiment, the first closure member 140 is part of or coupled to the refill head 120. However, dependent upon the location of the cavity 130, the location of the first closure member 140 may also be modified. The first closure member 140 is depicted in the closed state in FIG. 1 and in the open state in FIG. 4A. The first closure member 140 may be alterable between the closed and open states by manual interaction by a user or automatically via electrical operation. The exemplified embodiment illustrates the manual operation and will be discussed in greater detail below, but electrical operation may be used such that a user may simply press a button or otherwise actuate some type of actuation mechanism to cause the first closure member 140 to be altered from the closed state to the open state, and vice versa.

The first closure member 140 may comprise a door 141 that covers the first opening 131 when the first closure member 140 is in the closed state. The door 141 may have a first surface 142 that faces outward and is exposed when the first closure member 140 is in the closed state. The first closure member 140 may comprise a tab 143 that extends or protrudes from the first surface 142 of the door 141. The tab 143 may protrude from the door 141 so that it can be gripped by a user to alter the first closure member 140 between the closed and open states.

The body 101 may comprise a slot 128 within which the tab 143 may slide as the first closure member 140 is altered between the closed and open states. In the exemplified embodiment, the slot 128 is formed into the front surface 126 of the sleeve portion 121 of the refill head 120. The slot 128 may extend axially from the first opening 131 in the front surface 126 of the sleeve portion 121 of the refill head 120. In the exemplified embodiment, the slot 128 extends downwardly from the first opening 131 towards the gripping portion 111 of the handle portion 110. However, the invention is not to be so limited and the slot 128 may extend upwardly or laterally or diagonally or in any desired direction from the first opening 131. As should be appreciated, as the user grips the tab 143 and slides the tab 143 along the slot 128, the door 141 moves or slides relative to the first opening 131 to open/close the first opening 131 as discussed herein. Although in the exemplified embodiment the first closure member 140 slides relative to the refill head 120, in other embodiments the first closure member 140 may be attached to the refill head 120 via a hinge so that the first closure member 140 pivots relative to the refill head 120 like a conventional door. Other manners of movement of the first closure member 140 between the open and closed states may also be permissible within the scope of the invention claimed herein.

As noted above, in the exemplified embodiment the cavity 130 extends from the first opening 131 to the second opening 132. In other embodiments, the second opening 132 may be omitted and the cavity 130 may comprise only the first opening 131. However, in the exemplified embodiment whereby the cavity 130 comprises the second opening 132, the oral care implement 100 may comprise a second closure member 150 that is alterable between: (1) a closed state whereby the second closure member 150 closes the second opening 132 of the cavity 130; and (2) an open state whereby the second closure member 150 does not completely close the second opening 132 of the cavity 130 so that a passageway exists into the cavity 130 through the second opening 132. In the exemplified embodiment, the second closure member 150 is part of or coupled to the refill head 120. However, dependent upon the location of the cavity 130, the location of the second closure member 140 may also be modified. The second closure member 150 is depicted in the closed state in FIG. 2 and in the open state in FIG. 4A. The second closure member 150 may be alterable between the closed and open states by manual interaction by a user or automatically via electrical operation. The exemplified embodiment illustrates the manual operation and will be discussed in greater detail below, but electrical operation may be used such that a user may simply press a button or otherwise actuate some type of actuation mechanism to cause the second closure member 150 to be altered from the closed state to the open state, and vice versa.

The second closure member 150 may comprise a door 151 that covers the second opening 132 when the second closure member 150 is in the closed state. The door 151 may have a first surface 152 that faces outward and is exposed when the second closure member 150 is in the closed state. The second closure member 150 may comprise a tab 153 that extends or protrudes from the first surface 152 of the door 151. The tab 153 may protrude from the door 151 so that it can be gripped by a user to alter the second closure member 150 between the closed and open states.

The body 101 may comprise a slot 129 within which the tab 153 may slide as the second closure member 150 is altered between the closed and open states. In the exemplified embodiment, the slot 129 is formed into the rear surface 127 of the sleeve portion 121 of the refill head 120. The slot 129 may extend axially from the second opening 132 in the rear surface 127 of the sleeve portion 121 of the refill head 120. In the exemplified embodiment, the slot 129 extends downwardly from the second opening 132 towards the gripping portion 111 of the handle portion 110. However, the invention is not to be so limited and the slot 129 may extend upwardly or laterally or diagonally or in any desired direction from the second opening 132. As should be appreciated, as the user grips the tab 153 and slides the tab 153 along the slot 129, the door 151 moves or slides relative to the second opening 132 to open/close the second opening 132 as discussed herein. Although in the exemplified embodiment the second closure member 150 slides relative to the refill head 120, in other embodiments the second closure member 150 may be attached to the refill head 120 via a hinge so that the second closure member 150 pivots relative to the refill head 120 like a conventional door. Other manners of movement of the second closure member 150 between the open and closed states may also be permissible within the scope of the invention claimed herein.

In the exemplified embodiment, the sleeve portion 121 of the refill head 120 defines a sleeve cavity 105. That is, the sleeve portion 121 of the refill head 120 may be hollow to define the sleeve cavity 105. When the refill head 120 is coupled to the handle portion 110, the stem portion 112 of the handle portion 110 may nest within the sleeve cavity 105. In the exemplified embodiment, the distal portion 114 of the stem portion 112 where the at least one sensor 200 is located is axially aligned with the cavity 130. That is, an axis transverse to the longitudinal axis A-A will intersect the first and second openings 131, 132 of the cavity 130 and will also intersect the distal portion 114 of the stem portion 112, and more specifically the sensor 200 located therein or therealong. Since the cavity 130 is configured to receive a sample of the user's saliva, the sensor 130 being aligned with the cavity 130 allows the sensor 130 to obtain data useful for determining whether hemoglobin is present in the sample of the user's saliva.

The cavity 130 may be formed by a portion of the sleeve cavity 105 which extends between the first and second openings 131, 132. In other embodiments, particularly embodiments where the oral care implement 100 is a unitary construct, the cavity 130 may be a through-hole which extends between two openings 131, 132, a recess or pocket having just one opening, such as the opening 131, or the like.

The exact location of the at least one sensor 200 as shown in the drawings is not to be limiting of the invention in all embodiments. In particular, in other embodiments the at least one sensor 200 may be located proximal but adjacent to the cavity 130, with the sensor 200 facing in a distal direction towards the cavity 130. In this manner, despite not being in axial alignment with the cavity 130, the sensor 200 may nonetheless still be capable of taking measurements and obtaining data relevant to the presence or absence of hemoglobin/blood in a sample located in the cavity 130.

In the exemplified embodiment, the distal portion 114 of the stem portion 112 is located along or within the cavity 130 when the refill head 120 is coupled to the handle portion 110. The distal portion 114 of the stem portion 112 may form a collection surface on which the sample of the user's saliva may be placed. In some embodiments, the front surface of the distal portion 114 of the stem portion 112 may comprise a recess or a well within which the sample may be collected. This may help to maintain the sample of the user's saliva on the distal portion 114 of the stem portion 112 when the sensor 200 is operating to obtain data from the sample. In other embodiments, the distal portion 114 of the stem portion 112 may not extend into the cavity 130, but may instead be positioned just below and adjacent to the cavity 130. In such embodiments, the sample of the user's saliva may be located within the empty space within the cavity 130 while the sensor 200 may be directed towards the sample to take measurements and obtain data therefrom as described herein.

Referring to FIG. 5, the at least one sensor 200 will be described in greater detail in accordance with an embodiment of the present invention. The at least one sensor 200 is preferably small so that it can fit within the stem portion 112 and/or the head portion 122 of the oral care implement 100 as depicted in the figures. In the exemplified embodiment the at least one sensor 200 comprises a transmitter 210 and a receiver 202. Furthermore, in the exemplified embodiment the transmitter 210 comprises a first light source 211, a second light source 212, and a third light source 213. Although three light sources are depicted in the exemplified embodiment, the invention is not to be so limited in all embodiments. Specifically, in some alternative embodiments the transmitter 210 may include only the first and second light sources 211, 212 and not also the third light source 213. One example of the at least one sensor 200 is MAX30105 from Maxim Integrated, although the invention is not to be limited to this in all embodiments and other sensors could be used. In some embodiments, the transmitter 210 can be a broadband white light emitter. In such an embodiment, the receiver 220 may have multiple channels to detect reflected light at different wavelengths separately.

In the exemplified embodiment, the at least one sensor 200 is a singular structure that includes all of the features/components noted herein. However, the invention is not to be so limited and in some other embodiments the sensor may comprise multiple sensors that are independent and distinct from one another. For example, the sensor may include discrete light sources that are separate and apart from a receiver. Thus, the term sensor, as used herein, includes the situation where a single sensor having all of the necessary components is used and the situation where multiple sensors that in combination have all of the necessary components are used.

The first light source 211 is configured to emit light at a first wavelength, the second light source 212 is configured to emit light at a second wavelength that is different than the first wavelength, and the third light source is configured to emit light at a third wavelength that is different than the first and second wavelengths. For example, in one embodiment the first light source 211 is configured to emit red light having a wavelength in a range of 625-740 nm, more specifically 640-680 nm, and still more specifically approximately 660 nm. Furthermore, in one embodiment the second light source 212 is configured to emit green light having a wavelength in a range of 520-590 nm, more specifically 520-540 nm, and still more specifically between 525 nm and 530 nm. Further still, in one embodiment the third light source 213 is configured to emit infrared light having a wavelength in a range of 700 nm-1 micron, and more specifically 830-930 nm, and still more specifically 860-900 nm. In the exemplified embodiment, each of the first, second, and third light sources 211, 212, 213 are light emitting diodes, although other types of light sources may be used in the alternative. Thus, the transmitter 210 of the at least one sensor 200 may comprise multiple light sources such that each of the light sources transmits light at a different wavelength. The receiver 220 may be a broad spectrum light detector such that it can detect reflected light in all of the wavelengths mentioned herein (i.e., it can detect at least red, green, and infrared light).

While the description above describes three ranges of light (red light, green light, and infrared light), the invention is not to be so limited in all embodiments. In other embodiments, additional wavelengths and light sources may be used in the same or different wavelengths to enhance the measurement results being obtained.

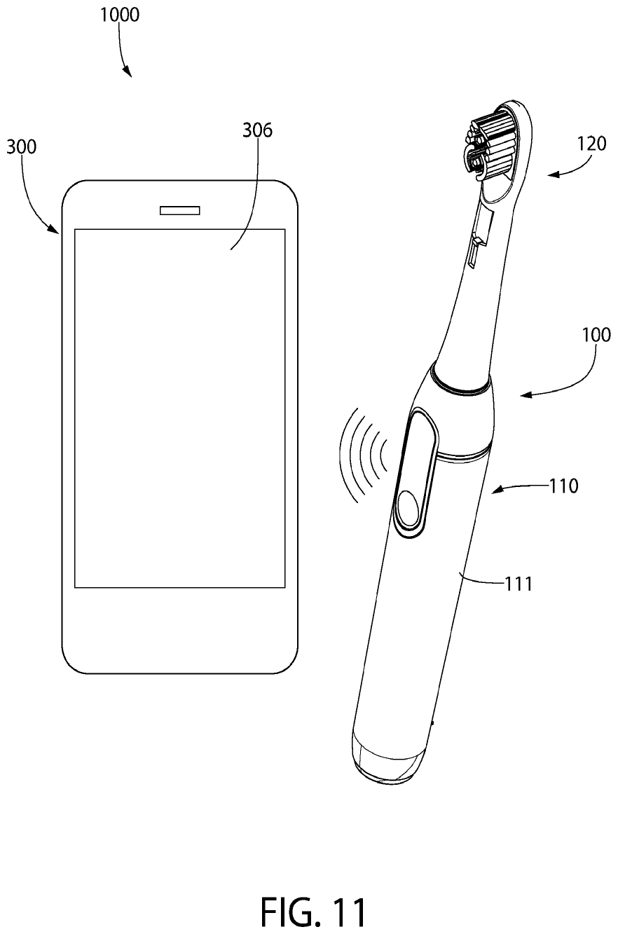

The at least one sensor 200 may be operably coupled to a processor 230 so that measurements, data, or other information detected and/or obtained by the at least one sensor 200 can be transmitted to the processor 230 as a signal for processing. The oral care implement 100 may comprise the processor 230, such that the processor 230 may be located within the handle portion 110 of the oral care implement 100. Alternatively, the processor 230 may be a part of a separate electronic device, such as a smart phone, which may be in operable communication with the oral care implement 100. This will be discussed further below with reference to FIGS. 11 and 12.

The processor 230 may pre-programmed with algorithms or otherwise work in association with software applications containing algorithms so that the processor 230 can perform various calculations using the information acquired by the at least one sensor 200 to determine whether hemoglobin, and hence also blood, is being detected by the at least one sensor 200. Specifically, the processor 230 may perform calculations and then, utilizing the pre-programmed algorithms, determine whether the results of those calculations indicate that hemoglobin/blood is present or not, and if so at what quantity.

Referring to FIGS. 6A and 6B, operation of the at least one sensor 200 will be described. FIG. 6A schematically depicts the at least one sensor 200 positioned so that that light emitted by the at least one sensor 200 can contact and be reflected by a sample of a user's saliva 250 located in the cavity 130 of the oral care implement 100. In FIG. 6A, the sample of the user's saliva 250 is devoid of any blood. FIG. 6B schematically depicts the at least one sensor 200 located positioned so that the light emitted by the at least one sensor 200 can contact and be reflected by a sample of the user's saliva 251 located in the cavity 130 of the oral care implement 100. In FIG. 6B, the sample of the user's saliva 251 may comprises blood. As discussed above, a toothpaste slurry is a liquid formulation that includes toothpaste and saliva. Where the disclosure refers to saliva, this may refer to plain saliva or saliva mixed with toothpaste, such as a toothpaste slurry. Furthermore, if there is blood in the mouth, this blood will mix with the liquid formulation and also form a part of the user's saliva and/or toothpaste slurry. Thus, the at least one sensor 200 is configured to detect whether there is blood in the sample of the user's saliva (e.g., the toothpaste slurry), which would in turn be indicative of bleeding occurring within the oral cavity (i.e., gum bleeding or the like).

Referring first to FIG. 6A, the at least one sensor 200 is performing a sensing operation on the sample of the user's saliva 250 that is devoid of any blood. Thus, the sample of the user's saliva 250 includes saliva and perhaps also toothpaste, but no blood. In this embodiment, the first light source 211 emits a first light 214 towards the sample of the user's saliva 250 at the first wavelength (e.g., red light), the second light source 212 emits a second light 215 towards the sample of the user's saliva 250 at the second wavelength (e.g., green light), and the third light source 213 emits a third light 216 towards the sample of the user's saliva 250 at the third wavelength (e.g., infrared light). In a complicated matrix such as that of saliva/toothpaste slurry, abrasive particles and air bubbles in the toothpaste slurry reflect light at different wavelengths at a similar efficiency. Thus, as shown in FIG. 6A, the first, second, and third lights 214, 215, 216 are all reflected off of the sample of the user's saliva 250 as a reflected portion of the first light 217, a reflected portion of the second light 218, and a reflected portion of the third light 219.

Referring to FIG. 6B, the at least one sensor 200 is performing a sensing operation on the sample of the user's saliva 251 that comprises blood. Thus, the sample of the user's saliva 251 includes toothpaste, saliva, and blood due to gum or other oral tissue surface bleeding. In this embodiment, the first light source 211 emits the first light 214 towards the sample of the user's saliva 251 at the first wavelength (e.g., red light), the second light source 212 emits a second light 215 towards the sample of the user's saliva 251 at the second wavelength (e.g., green light), and the third light source 213 emits a third light 216 towards the sample of the user's saliva 251 at the third wavelength (e.g., infrared light). Due to its strong red color, hemoglobin in red blood cells will strongly absorb green light, while reflecting a majority of red and infrared light back.

Thus, as shown in FIG. 6B, the first and third light 214, 216 are reflected from the sample of the user's saliva 251 as a reflected portion of the first light 217 and a reflected portion of the third light 219. However, the second light 215 (which is the green light in the exemplified embodiment) is absorbed by the sample of the user's saliva 251. In FIG. 6B, it is illustrated such that none of the second light 215 is reflected back to the receiver 220 of the at least one sensor 200. However, in actual practice some of the second light 215 is reflected back, but it has a reduced intensity as compared to the amount of the second light 215 that is reflected back from the sample of the user's saliva 250 of FIG. 6A that is devoid of blood due to the hemoglobin absorbing some of the second light 215. Thus, the second light 218 that is reflected from the sample of the user's saliva 250 that is devoid of blood has a greater intensity than the second light 218 that is reflected from the sample of the user's saliva 251 that comprises blood due to the hemoglobin in the blood absorbing some of the second light 215.

Thus, at desired times, the at least one sensor 200 transmits the first, second, and third lights 214, 215, 216 into the cavity 130. The first, second, and third lights 214, 215, 216 contact the sample of the user's saliva 250, 251 in the cavity 130 and reflected portions of the first, second, and third lights 217, 218, 219 are received by the receiver 220 of the at least one sensor 200. The intensity of the reflected portions of the first and third lights 217, 219 (red and infrared light) are relatively unchanged regardless of whether or not the sample of the user's saliva comprises blood. However, the intensity of the reflected portion of the second light 218 (green light) is greater when the sample of the user's saliva does not comprise blood than when it does. Thus, the at least one sensor 200 generates a first signal indicative of a first intensity of the reflected portion of the first light 217, a second signal indicative of a second intensity of the reflected portion of the second light 218, and a third signal indicative of the third intensity of the reflected portion of the third light 219.

Because the intensity of the reflected portion of the second light 218 is reduced when there is blood in the sample of the user's saliva while the intensity of the reflected portions of the first and third light 217, 219 remain substantially the same regardless of whether or not there is blood in the sample of the user's saliva, the ratio of the reflected light intensities can be used to identify and quantify hemoglobin/blood. Thus the processor 230 may have an algorithm that can calculate the ratios and determine whether or not (and how much) hemoglobin and blood is present.

Due to their operable coupling, the first, second, and third signals (although the third signal could be omitted because the system could operate just as well with only red or infrared light and green light being transmitted by the at least one sensor 200) is transmitted from the at least one sensor 200 to the processor 230. The processor 230 is equipped with algorithms instructing it to perform calculations with the first, second, and third signals to assist in determining whether hemoglobin/blood is in the toothpaste slurry. Specifically, the processor 230 is configured to calculate a ratio of the first intensity of the first light 217 to the second intensity of the second light 218 and/or a ratio of the third intensity of the third light 219 to the second intensity of the second light 218. As should be appreciated, if there is hemoglobin/blood in the toothpaste slurry, the intensity of the reflected portion of the second light 218 is less than if there is no hemoglobin in the toothpaste slurry. Thus, if there is hemoglobin/blood in the toothpaste slurry, the ratio of the first intensity of the first light 217 to the second intensity of the second light 218 and the ratio of the third intensity of the third light 219 to the second intensity of the second light 218 is increased as compared to the situation where there is no hemoglobin/blood in the toothpaste slurry (due to the denominator in the ratio calculation being reduced). Using this understanding and the developed algorithms, the processor 230 can make a determination as to whether there is hemoglobin/blood in the toothpaste slurry, and if so, how much.

For example, the processor 230 or algorithm may be set with a predetermined threshold for the various ratios that are calculated so that upon the ratio exceeding the predetermined threshold, the processor 230 will be informed that hemoglobin/blood has been detected. Thus, the processor 230 can be programmed so that if the ratio of the first intensity of the first light 217 to the second intensity of the second light 218 exceeds a first predetermined threshold and/or the ratio of the third intensity of the third light 219 to the second intensity of the second light 218 exceeds a second predetermined threshold, hemoglobin/blood is present.

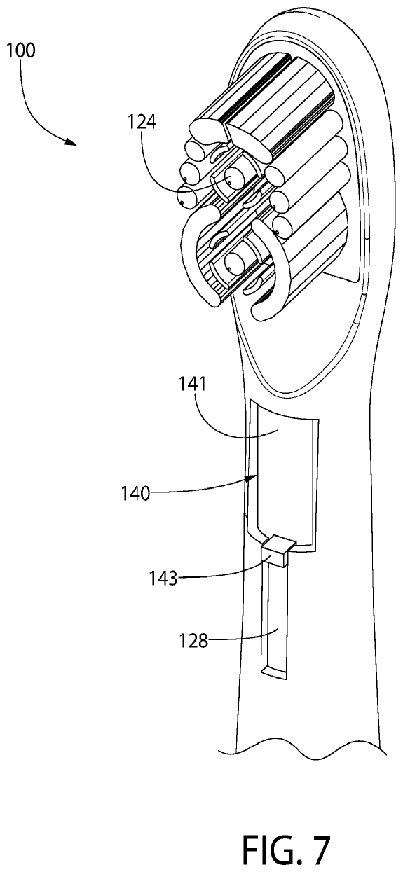

Referring to FIGS. 7-10, operation and use of the oral care implement 100 to obtain data useful for determining whether hemoglobin is present in the user's saliva will be described. FIG. 7 illustrates the oral care implement 100 with the first closure member 140 in the closed state. Generally, the first closure member 140 will remain in the closed state when the user brushes his or her teeth with the tooth cleaning elements 124 of the oral care implement 100. This prevents the user's saliva and/or toothpaste slurry from entering into the cavity 130 during toothbrushing. In some embodiments of the present invention, it is desirable to test the user's saliva/toothpaste slurry for the presence of blood after toothbrushing is complete, or at other times as desired by a user which are not during a toothbrushing session. For example, the user may wish to test their saliva for blood after flossing. By keeping the first closure member 140 closed during toothbrushing, the user's saliva will not pass into the cavity 130 during toothbrushing so the user can obtain a good reading of blood in the saliva only after toothbrushing is complete. Of course, if the user desires to obtain information regarding the presence of blood/hemoglobin in the saliva/toothpaste slurry during toothbrushing, the user may wish to leave the first closure member 140 in the open state during toothbrushing so that the user's saliva/toothpaste slurry may enter into the cavity 130 during the toothbrushing session.

While not depicted in FIG. 7, at this step the second closure member 150 may also be in the closed state. In some embodiments, if the first closure member 140 is in the open state during toothbrushing, the second closure member 150 will be in the closed state so that saliva/toothpaste slurry can gather in the cavity 130. In other embodiments, the first and second closure members 140, 150 may remain in the closed state during toothbrushing so that saliva samples are only introduced into the cavity 130 after toothbrushing has been completed or at other times that are not during a toothbrushing session.

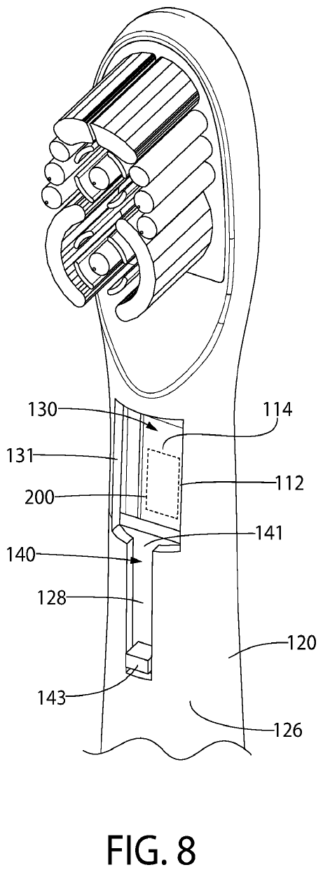

Referring to FIG. 8, the oral care implement 100 is illustrated with the first closure member 140 having been altered from the closed state shown in FIG. 7 to the open state. In the exemplified embodiment, this is achieved by sliding the second closure member 140 relative to the refill head 120 (or the body 101 generally) of the oral care implement 100. In particular, the user may grip the tab 143 of the first closure member 140 and slide the tab 143 along the slot 128 formed into the front surface 126 of the refill head 120 (or body 101). As the tab 143 slides downwardly within the slot 128, the door 141 slides downwardly to expose the first opening 131 of the cavity 130. As seen in FIG. 8, the first opening 131 is exposed and provides a passageway into the cavity 130 from the exterior. In FIG. 8, the distal portion 114 of the stem portion 112 is visible in the cavity 130 due to the axial alignment of the distal portion 114 of the stem portion 112 with the cavity 130 as described above. Furthermore, the sensor 200 is aligned with the first opening 131. As discussed above, the stem portion 112 may terminate proximal to the cavity 130 in some embodiments. In FIG. 8, the second closure member 150 may remain in the closed state.

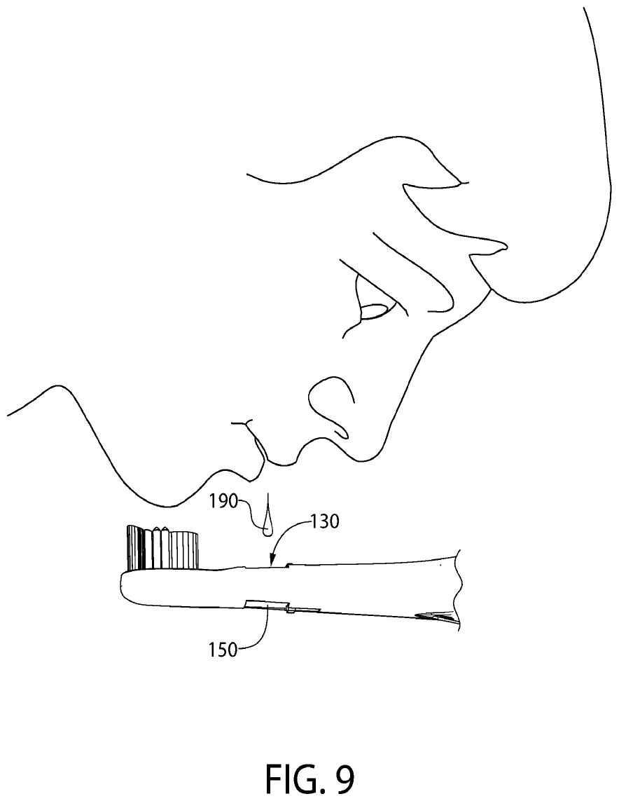

Next, referring to FIG. 9, with the first closure member 140 in the open state and the second closure member 150 in the closed state, a user may introduce a sample of the user's saliva (or a sample of a toothpaste slurry) 190 into the cavity 130 of the oral care implement 100. In the exemplified embodiment, this is achieved by the user spitting directly into the cavity 130. However, the invention is not to be so limited and the user may otherwise scoop the sample out of the user's mouth and introduce it into the cavity 130. The user may user a syringe to collect the sample from the user's mouth and then expel the sample into the cavity 130. Thus, it should be appreciated that there are various techniques that may be used to introduce the sample of the saliva/toothpaste slurry into the cavity 130. By having the first closure member 140 in the open state, the sample 190 can be introduced through the first opening 131 and into the cavity 130. By having the second closure member 150 in the closed state, the sample 190 is held in the cavity 130 and cannot pass through the second opening 132 (which may be omitted in some embodiments, as discussed above).

Once the sample 190 has been introduced into the cavity 130, the sensor 200 may be activated to emit the light into the cavity 130 and receive the reflected portions of the light, as described in detail above with reference to FIGS. 5, 6A, and 6B. The sensor is able to collect data or other information which may be used by a processor to determine whether hemoglobin is present in the user's saliva which is located in the cavity 130. The discussion above is applicable to this process and is not repeated here in the interest of brevity.

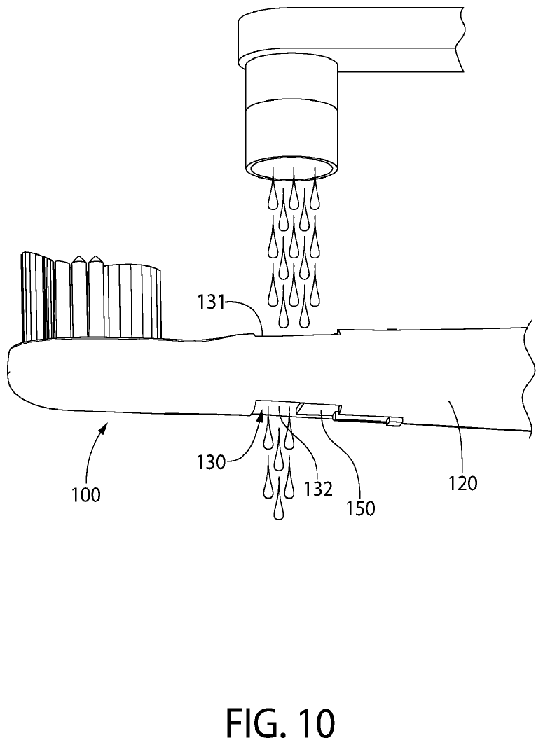

Once the at least one sensor 200 has performed its measurement function and obtained data useful for determining whether hemoglobin is present in the sample 190, the user may rinse out the cavity 130. Referring to FIG. 10, in one embodiment the user may alter the second closure member 150 from the closed state to the open state to expose the second opening 132. The first and second closure members 140, 150 may be independently alterable between their respective open and closed states. The user may then rinse out the cavity 130 by flowing water or a cleaning solution through the cavity 130. The water or cleaning solution may flow through the first opening 131, through the cavity 130, and then out through the second opening 132 (or may flow in the opposite direction). In other embodiments, the user may detach the refill head 120 from the handle portion 110 and then rinse out the cavity 130.

Referring to FIGS. 11 and 12, a system 500 for detecting blood in an oral cavity. The system 500 includes the oral care implement 100 and a portable electronic device 300 that are in operable communication with one another. The oral care implement 100 is the one described above with reference to FIGS. 1-4, and thus the description above is applicable.

The oral care implement 100 may comprise an electronic circuit that may include the processor 230, the power source 199, the sensor 200, a memory device 260 (which could alternatively be a part of the processor 230 in some embodiments), a Bluetooth module 261, and an indicator 262. However, it may be possible to omit the processor 230, the memory device 260, and/or the indicator 262 in some embodiments. Thus, in some embodiments the electronic circuit of the oral care implement 100 may comprise only the sensor 200 (which includes the transmitter 210 and a receiver 220) and the power source 199, and the Bluetooth module 261. The oral care implement 100 may also include a motor 263 in instances where the oral care implement 100 is a powered toothbrush.

The reason that the oral care implement 100 may not include the processor 230, the memory device 260, and the indicator 262 (although it may include any of one or more of these components in some embodiments) is because these components may be included as a part of the portable electronic device 300 that is in communication with the oral care implement 100. Specifically, the portable electronic device 300 may be a smart phone, a tablet, a computer, or a similar device that includes a processor 301, a memory 302, a user interface 303, a blood detection software application 304, and a Bluetooth module 305. The portable electronic device 300 may also include a display 306 (which can be the same as the user interface 303 or distinct from the user interface 303). In the exemplified embodiment, the processor 301 of the portable electronic device 300 is in operable communication with the sensor 200 of the oral care implement 100 via Bluetooth due to the incorporation of the Bluetooth module 261 in the oral care implement 100 and the Bluetooth module 305 in the portable electronic device 300 (when the oral care implement 100 and the portable electronic device 300 are in sufficiently close proximity to one another so as to allow for such a Bluetooth connection). Of course, Bluetooth is merely one exemplary way that the oral care implement 100 and the portable electronic device 300 can be in communication. In other embodiments, there may be a wired connection between the oral care implement 100 and the portable electronic device 300 or they may communicate using other wireless protocols (infrared wireless communication, satellite communication, radio, microwave, Zigbee, Z-wave, or the like). Of course, the sensor 200 may have a built-in microcontroller in some embodiments.

In this embodiment, the sensor 200 will operate as described above. Thus, the transmitter 210 of the sensor 200 comprises multiple light sources that emit light at different wavelengths and the receiver 220 of the sensor 200 receives reflected light. The sensor 200 then generates signals indicative of the intensities of the various reflected lights. In this embodiment the signals are then transmitted, via Bluetooth or otherwise, from the sensor 200 of the oral care implement 100 to the processor 301 of the portable electronic device 300. The transmission of these signals from the sensor 200 of the oral care implement 100 to the processor 301 of the portable electronic device 300 may occur so long as the oral care implement 100 and the portable electronic device 300 are in operable communication (either via Bluetooth or other wireless technologies or through a wired connection).

In an alternative embodiment, the processing may all take place within the oral care implement 100. In such embodiments, the processor 230 of the oral care implement 100 may receive the signals from the sensor 200 and determine whether hemoglobin/blood is present in the sample as discussed herein. In such an embodiment, the Bluetooth module 261 may be omitted if there is no reason to transmit information from the oral care implement 200 to an external device like the portable electronic device 300. Furthermore, in such an embodiment the oral care implement 100 may comprise the indicator 262, which may be a visual, haptic, or audible indicator component. For example, the indicator 262 may flash a color to indicate when hemoglobin is detected, or when hemoglobin is detected above a certain threshold. The indicator 262 may not flash, or may flash a different color when no hemoglobin is detected or the hemoglobin levels are below a threshold. Alternatively, the indicator 262 may vibrate or make a particular sound to indicate the presence or absence of hemoglobin in the tested sample.

In some embodiments, the information associated with the signals and the information detected by the sensor 200 may be stored in the memory 260 and/or processor 230 of the oral care implement 100 initially and then transferred to the processor 301 of the portable electronic device 300 in batches. Thus, data or information corresponding to a plurality of different toothbrushing sessions may initially be stored in the memory 260 and/or processor of the toothbrush 230. This can be useful in instances in which the user brushes his/her teeth at a time that the oral care implement 100 is not in operable communication with the portable electronic device 300. In this way, the oral care implement 100 will initially store all of the data, and then once the oral care implement 100 becomes operably coupled to the portable electronic device 300, the data can be transmitted to the portable electronic device 300 for further processing as described herein (either automatically or in response to manual user input). In such embodiments, the processor 230 may be able to process the data so that the processor 230 can activate the indicator 262 when it determines that hemoglobin/blood is present in the oral cavity or toothpaste slurry.

As noted above, the portable electronic device 300 may have a blood detection software application 304 downloaded thereon. Thus, a user may open the blood detection software application 304 and put the oral care implement 100 into operable (wireless or wired) communication with the portable electronic device 300. Alternatively, the operable coupling between the oral care implement 100 and the portable electronic device 300 may cause the blood detection software application 304 to automatically launch on the portable electronic device 300. In such a situation, as the sensor 200 of the oral care implement 100 is gathering information/data related to the intensity of the reflected light, it will transmit this data/information to the processor 301 of the portable electronic device 300. In some embodiments, this transmission of data/information from the sensor 200 to the processor 301 may occur automatically so long as the oral care implement 100 and the portable electronic device 300 are in operable communication with one another. Of course, as noted above, this data/information can alternatively (or additionally) be stored locally on the memory device 260 of the oral care implement 100 and then transmitted to the portable electronic device 300 in batches. The processor 301 of the portable electronic device 300 may make this data/information available to a user in various ways on the portable electronic device 300 using the blood detection software application 304.

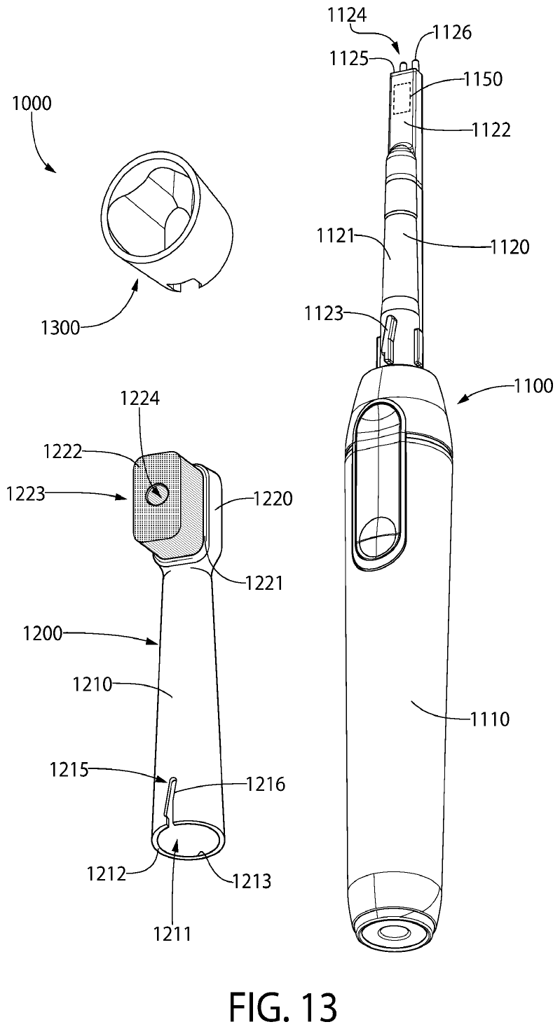

Referring to FIG. 13, an oral care system 1000 is illustrated in accordance with an embodiment of the present invention. The oral care system 1000 generally comprises a handle 1100, a refill head 1200, and a collection cup 1300. The refill head 1200 is detachably coupled to the handle 1100. The refill head 1200 may be detachably coupled to the handle 1000 to perform an oral care function, such as tooth cleaning. When the refill head 1200 is coupled to the handle 1100, the assembled device may be referred to as an oral care implement or a toothbrush. The collection cup 1300 is also detachably coupled to the handle 1100. The collection cup 1300 may be coupled to the handle to perform a sensing, testing, or measurement function, such as testing a sample of saliva or toothpaste slurry for the presence of hemoglobin/blood. In accordance with the exemplified embodiment, only one of the refill head 1200 and the collection cup 1200 may be coupled to the handle 1100 at a time. In some embodiments, the oral care system 1000 may comprise the handle 1100 and the collection cup 1300 only.

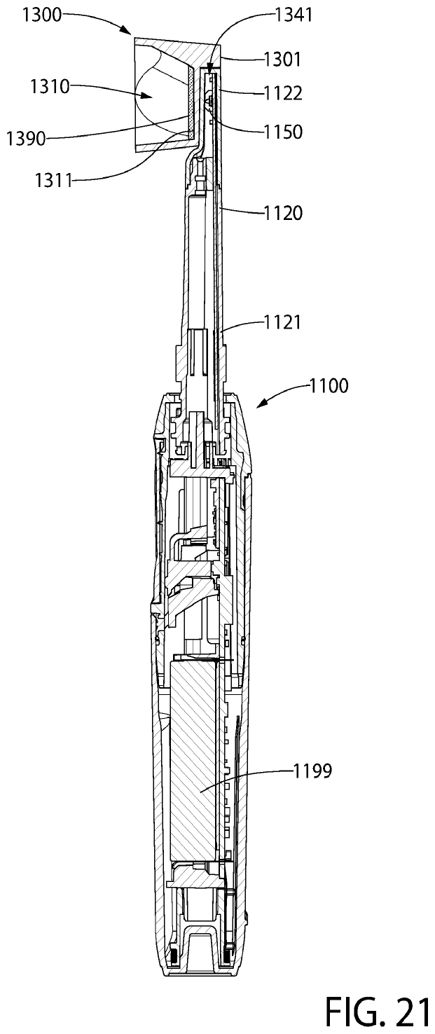

The handle 1100 comprises a gripping portion 1110 and a stem portion 1120 that extends from a distal end of the gripping portion 1110. The gripping portion 1110 may comprise an internal cavity that houses various electronic components such as a power source and the like (best seen in FIG. 21). The gripping portion 1110 may be gripped and held by the user during use. The stem portion 1120 may comprise an internal cavity that houses at least one sensor 1150. The sensor 1150 may be identical to the sensor 200 described above, and thus the description of the structure and function of the sensor 200 is applicable to the sensor 1150 and will not be described herein in the interest of brevity. In particular, FIGS. 5, 6A, and 6B and the disclosure set forth herein related to those figures is applicable to the sensor 1150.

The stem portion 1120 of the handle 1100 may comprise a proximal portion 1121 located adjacent to the gripping portion 1110 and a distal portion 1122 located furthest from the gripping portion 1110. The sensor 1150 may be located within, or along, the distal portion 1122 of the stem portion 1120. The stem portion 1120 may comprise a first connection feature 1123 and a second connection feature 1124. The proximal portion 1121 of the stem portion 1120 may comprise the first connection feature 1123. In the exemplified embodiment, the first connection feature 1123 may be a protrusion or boss located along the proximal portion 1121 of the stem portion 1120. However, the invention is not to be so limited and the first connection feature 1123 may take on other forms, including being an aperture, a slot, screw threads, a snap-fit feature, a friction fit feature, or the like, to name a few. The first connection feature 1123 of the stem portion 1120 of the handle 1100 is configured to engage or mate with a connection feature of the refill head 1200 discussed below to facilitate the connection between the refill head 1200 and the handle 1100.

The distal portion 1122 of the stem portion 1120 may comprise the second connection feature 1124. The distal portion 1122 of the stem portion 1120 may terminate in a distal end 1125 of the stem portion 1120. The second connection feature 1124 may comprise one or more protrusions 1126 that protrude from the distal end 1125 of the stem portion 1120. In other embodiments, the second connection feature 1124 may be formed by the shape of the distal portion 1122 of the stem portion 1120 (the protrusions 1126 may be omitted in some embodiments). In still other embodiments, the second connection feature 1124 may comprise the shape of the distal portion 1122 in combination with the one or more protrusions 1126. The second connection feature 1124 may take on other forms in other embodiments, such as being a feature of a snap-fit or friction fit connection, screw threads, or the like. The second connection feature 1124 may be separate and distinct from the first connection feature 1123.

The refill head 1200 comprises a sleeve portion 1210 and a head portion 1220. The sleeve portion 1210 comprises a sleeve cavity 1211 within which the stem portion 1120 of the handle 1100 is located when the refill head 1200 is coupled to the handle 1100. The sleeve portion 1210 comprises a bottom end 1212 having an opening 1213 that provides a passageway into the sleeve cavity 1211. The refill head comprises a third connection feature 1215. In the exemplified embodiment, the sleeve portion 1210 of the refill head 1200 comprises the third connection feature 1215. Furthermore, in the exemplified embodiment the third connection feature 1215 comprises an aperture 1216 formed through the sleeve portion 1210. The aperture 1216 may extend from the bottom end 1212 upwardly towards the head portion 1220. The aperture 1216 of the third connection feature 1215 may be configured to receive the protrusion of the first connection feature 1123 of the handle 1100 to facilitate the coupling between the refill head 1200 and the handle 1100. In alternative embodiments, the first connection feature 1123 may be an aperture and the third connection feature 1215 may be a protrusion. In still other embodiments, the first and third connection features 1123, 1215 may take on other forms that allow the first and third connection features 1123, 1215 to engage one another to facilitate the coupling of the refill head 1200 to the handle 1100, such as screw threads, friction fit, snap-fit, etc. The idea is that there is some engagement or contact or mating between the first and third connection features 1123, 1215 which prevents the refill head 1200 from simply slipping off the handle 1100 if oriented upside-down, for example. Thus, the first and third connection features 1123, 1215 should create some engagement between the refill head 1200 and handle 1100 which helps to hold the refill head 1200 onto the handle 1100 when they are attached. The connection features may be as simple as the shape of the stem 1120 and the internal cavity 1211 which creates a friction fit.

The head portion 1220 of the refill head 1200 comprises a front surface 1221 having tooth cleaning elements 1222 located thereon and protruding therefrom. The details of the structure, material, and pattern of the tooth cleaning elements 1222 is the same as the provided above with reference to the tooth cleaning elements 124, with the exceptions set forth herein. In accordance with the exemplified embodiment, the tooth cleaning elements 1222 form a cleaning element field 1223. Furthermore, the cleaning element field 1223 defines an opening or cavity 1224, which is formed by a portion of the head portion 1220 not having any cleaning elements 1222 extending therefrom. Thus, there is a portion of the head portion 1220 that is devoid of any cleaning elements 1222, and this portion is surrounded by the cleaning elements 1222 to form the cavity 1224 within the cleaning element field 1223. Of course, in other embodiments the spacing between the cleaning elements 1222 of the cleaning element field 1223 may be different than that which is shown such that there is no cavity per se, but still so that a gap in the cleaning element field 1223 remains.

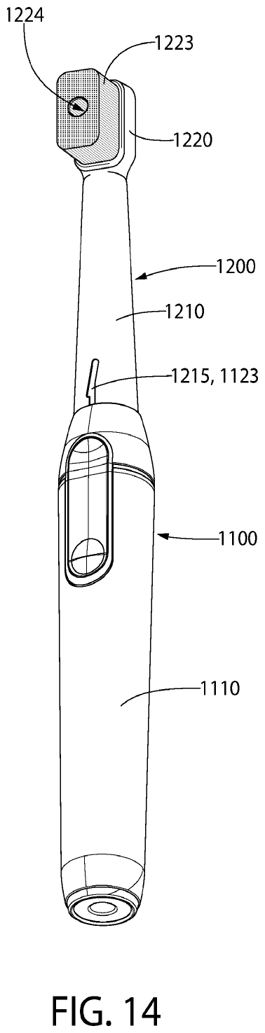

Referring briefly to FIG. 14, the refill head 1200 is illustrated coupled to the handle 1110. The stem 1120 nests within the sleeve cavity 1211 of the refill head 1200. The proximal and distal portions 1121, 1122 may nest within the sleeve cavity 1211. In some embodiments, the proximal portion 1121 may nest within the sleeve portion 1210 of the refill head 1200 and the distal portion 1122 of the stem 1120 may nest within an interior of the head portion 1220 of the refill head 1200. When the refill head 1220 is coupled to the handle 1110, the cavity 1224 in the bristle field 1223 may be aligned with the sensor 1150. As such, the sensor 1150 may be configured to emit light into the user's oral cavity during toothbrushing by emitting the light through the cavity 1224. In some embodiments, the head portion 1220 may be transparent to allow the light emitted from the sensor 1150 to pass through the head portion 1220. Using this structure, the sensor 1150 may be configured to obtain data useful for determining whether hemoglobin is present in the user's oral cavity during a toothbrushing session. That is, while the user brushes the teeth or otherwise cleans the oral cavity with the refill head 1200, the sensor 1150 may emit and receive light as described herein to obtain data regarding hemoglobin/blood being present or absent from the oral cavity. Additional details about this are described in United States Patent Publication No. 2022/0257968, which was previously incorporated herein by reference.

Alternatively (or in addition, the sensor 1150 (or some additional sensor) may be configured to perform additional or alternative functions when the refill head 1200 is coupled to the handle 1110. For example, the sensor 1150 or some additional or alternative sensor may be configured to detect or measure the presence or absence of the oral cavity, the presence e or lack thereof of an oral malady (i.e., plaque, gum disease, etc.) on the oral cavity, or the like. When the refill head 1220 is coupled to the handle 1110, the sensor, or some additional or alternative sensor, may be configured to perform any of one or more functions described in U.S. Pat. No. 11,406,480, the entirety of which is incorporated herein by reference.

In other embodiments, the cleaning element field 1223 may not have the cavity 1224 and the data may not be obtained during the toothbrushing session. Instead, the oral care system 1000 may be used to test for hemoglobin/blood in a sample of the user's saliva after completion of a toothbrushing session, for example using the collection cup 1300 described below. In other embodiments, the oral care system 1000 may be capable of testing for blood/hemoglobin during a toothbrushing session as described herein and also after a toothbrushing session or at any other desired time using the collection cup 1300. Data from the sensor 1150 may be processed internally within the oral care implement (or the handle 1100 thereof) and/or the data may be processed by an external electronic device, such as a mobile phone, tablet, computer, or the like, as described above with reference to FIGS. 11 and 12. The discussion of the processing of the data provided above is entirely applicable to the embodiment of the oral care system 1000.

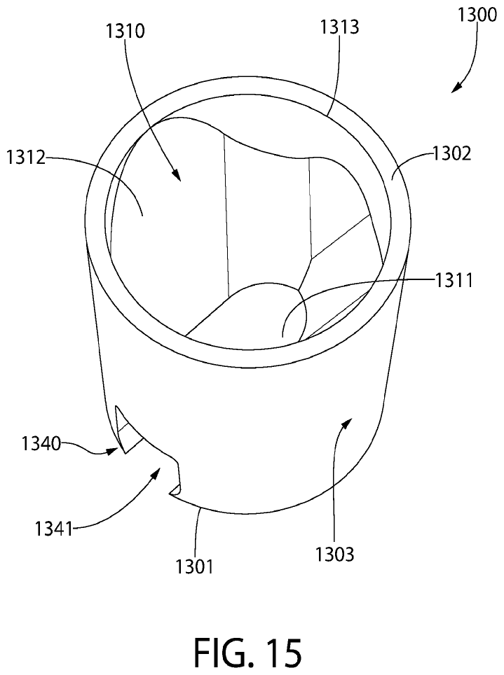

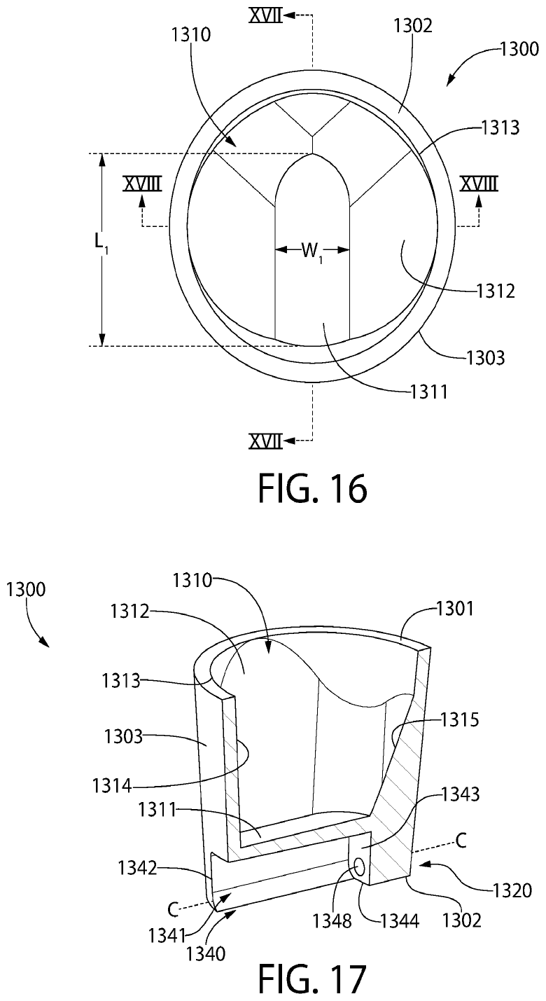

Referring to FIGS. 15-18A, the collection cup 1300 will be further described. The collection cup 1300 may be formed from glass, although this is not required and the collection cup 1300 may be formed from other materials, such as plastic. The collection cup 1300, or at least portions thereof which are aligned with the sensor 1150 when the collection cup 1300 is coupled to the handle 1100, may be transparent or light transmissive so that light emitted by the sensor 1150 may pass through the collection cup 1300. The collection cup 1300 comprises a bottom end 1301, a top end 1302, an outer surface 1303, and a collection cavity 1310 having a top opening 1313 at the top end 1302. The collection cavity 1310 is defined by a floor 1311 and an inner surface 1312 of the body of the collection cup 1300 that extends from the floor 1311 to the top end 1302 of the collection cup 1300. The collection cavity 1310 extends along a cavity axis B-B moving in a direction from the floor 1311 to the top end 1302. The collection cup 1300 further comprises a base portion 1320 located between the floor 1311 of the collection cavity 1310 and the bottom end 1302 of the collection cup 1300.

The collection cup 1300 comprises a fourth connection feature 1340 that is configured to engage with the second connection feature 1124 of the handle 1100 to facilitate the connection between the collection cup 1300 and the handle 1100, which will be described in greater detail below. The fourth connection feature 1340 may comprise a channel 1341 formed in the base portion 1320. The channel 1341 may extend from an opening 1342 in the outer surface 1303 of the collection cup 1300 to an end wall 1343. In other embodiments, the channel 1341 may extend from one opening in the outer surface 1303 to another opening in the outer surface, rather than having the end wall 1343. In the exemplified embodiment, the channel 1341 comprises an opening 1344 in the bottom end 1301 of the collection cup 1300 along its full length. However, the invention is not to be so limited in all embodiments and the channel 1341 could have a floor rather than extending through to the bottom end 1301.

The channel 1341 may be elongated in a direction from the opening 1342 to the end wall 1343 along a channel axis C-C. The channel axis C-C may be oriented perpendicularly relative to the cavity axis B-B. The channel 1341 may be defined by a first sidewall 1345 located on a first side of the channel axis C-C, a second sidewall 1346 located on a second side of the channel axis C-C, and a roof 1347 which extends between the first and second sidewalls 1345, 1346. As noted, the channel 1341 could also have a floor, although in the exemplified embodiment the channel 1341 extends through to the opening 1344 in the bottom end 1302 of the collection cup 1300. The roof 1347 of the channel 1341 may be generally aligned with the floor 1311 of the collection cavity 1310. The first and second sidewalls 1345, 1346 may comprise linear portions and inturned or curved portions, with the inturned or curved portions being located between the linear portions and the bottom end 1301 of the collection cup 1300. The inturned or curved portions may curve inwardly towards one another to form a locking feature that helps to retain the distal portion 1122 of the stem 1120 of the handle 1100 in the channel 1341 when the collection cup 1300 is coupled to the handle 1100. Other shapes and structures for the channel 1341 and its boundaries may be used in other embodiments.

In the exemplified embodiment, there are two holes 1348 in the end wall 1343. Each of the holes 1348 is configured to receive one of the protrusions 1126 of the handle 1100 when the collection cup 1300 is coupled to the handle 1100. Specifically, when the collection cup 1300 is coupled to the handle 1100, the distal portion 1122 of the stem 1120 nests within the channel 1341 and the protrusions 1126 nest within the holes 1148. The protrusions 1126 and holes 1148 could be omitted in other embodiments and the nesting of the distal portion 1122 of the stem 1120 within the channel 1341 may adequately couple the collection cup 1300 to the handle 1100. The collection cup 1300 may be slidably coupled to the handle 1100 by sliding the distal portion 1122 of the stem 1120 into the channel 1341 in the direction of the channel axis C-C.

The channel 1341 may be located directly beneath and in alignment with the floor 1311 of the collection cavity 1310. Stated another way, the floor 1311 may overlie the channel 1341. In some embodiments, any axis parallel to the cavity axis B-B which intersects the floor 1311 of the collection cavity 1310 will also intersect the channel 1341. As such, when the collection cup 1300 is coupled to the handle 1100, the sensor 1150 which is located along or within the distal portion 1122 of the stem 1120 will be located below and in alignment with the floor 1311 of the collection cavity 1310. That is, an axis parallel to the cavity axis B-B which intersects the sensor 1150 will also intersect the floor 1311. By having the sensor 1150 aligned with the floor 1311 of the collection cavity 1310, the sensor 1150 will be able to perform its testing/measurement/sensing function on saliva samples held in the collection cavity 1310.

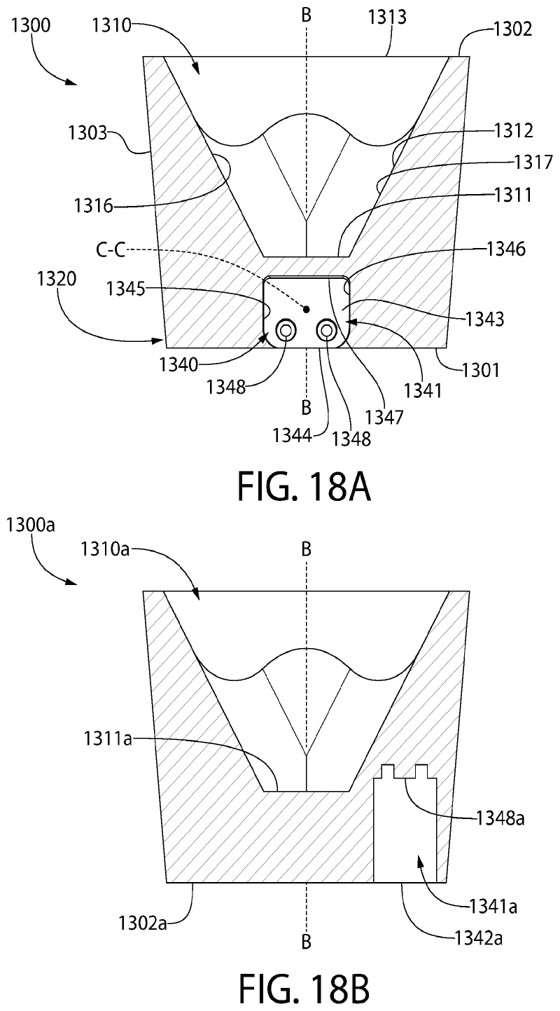

The inner surface 1312 of the body of the collection cup 1300 is angled towards the cavity axis B-B so that the floor 1311 of the collection cavity 1310 has a smaller footprint or area than the top opening 1313 of the collection cavity 1310. Different portions of the inner surface 1312 of the body of the collection cup 1300 may be oriented at different angles relative to the cavity axis B-B. For example, as seen in FIG. 17, a front-most portion 1314 of the inner surface 1312 is minimally angled relative to the cavity axis B-B, and may even be oriented parallel to the cavity axis B-B. A rear-most portion 1315 of the inner surface 1312 has a top portion which is linear and a bottom portion which is angled relative to the cavity axis B-B at a much greater angle than the front-most portion 1314. Then, as best seen in FIG. 18A, side portions 1316, 1317 of the inner surface 1312 are oriented at the same angle relative to the cavity axis B-B.

Due to the various angles of the inner surface 1312 of the sidewall as described herein, the floor 1311 of the collection cavity 1310 may be in the shape of a capsule or pill or a two-dimensional spherocylinder. The capsule or pill or spherocylinder shape may have one of its rounded ends cut off to form the final shape of the floor 1311, although this is not required in all embodiments. The floor 1311 may be deemed to have an arch shape in some embodiments. As such, the floor 1311 of the collection cavity 1310 has an area which is much smaller than that of the top opening 1313. Creating a small floor provides a greater likelihood that a sample of saliva introduced into the collection cavity 1310 will cover the entire floor, which may create the environment for better measurement results from the sensor 1150.

The top opening 1313 of the collection cavity 1310 may have an area of between 200 mm² and 500 mm², more specifically between 300 mm² and 450 mm², more specifically between 330 mm² and 400 mm², and more specifically between 350 mm² and 370 mm². In some embodiments, the area of the floor 1311 may be between 40 mm² and 150 mm², more specifically between 50 mm² and 120 mm², still more specifically between 60 mm² and 100 mm², and still more specifically between 80 mm² and 100 mm². In some embodiments, a ratio of the area of the top opening 131 to the area of the floor 1311 may be in a range of 3:1 and 5:1, more specifically 3.5:1 and 4.5:1, and still more specifically 3.8:1 and 4.2:1. By forming the floor 1311 with a small footprint, it is ensured that the saliva or toothpaste slurry which is held in the collection cavity 1310 will cover the floor 1311, which will enable the sensor 1150 to take better measurements when checking for blood/hemoglobin in the sample.

The floor 1311 may have a length L1 and a width W1. The length L1 may be greater than the width W1. A ratio of the length L1 to the width W1 may be between 1.5:1 and 3.5:1, more specifically 1.8:1 and 3.0:1, and still more specifically 2.0:1 and 2.5:1.

Referring briefly to FIG. 18B, a collection cup 1300a is illustrated. The collection cup 1300a is identical to the collection cup 1300, except with regard to the location/orientation of the channel 1341a. In particular, in this embodiment the channel 1341a is elongated in a direction which is parallel to the inner surface of the collection cup 1300 moving from an opening 1342a in the bottom end 1302a of the collection cup 1300a to an end wall 1343a. When the collection cup 1300 is coupled to the handle 1100 and contains a sample, the handle 1100 may be held generally horizontally (plus or minus) 20° to prevent the sample from spilling out. When the collection cup 1300a is coupled to the handle 1100 and contains a sample, the handle 1100 is held generally vertically (plus or minus) 20° to prevent the sample from spilling out. Otherwise, the collection cup 1300a and the collection cup 1300 are identical. That is, the only difference between the two is the position and orientation of the channels 1341, 1341a. Moreover, the orientation of the channel 1341a may be modified depending on the shape of the inner surface of the collection cup 1300a. That is, if one of the sidewalls of the inner surface of the collection cup 1300a is vertical, then the channel 1341a could also be vertical (i.e., parallel to the axis B-B) instead of being oriented at an angle relative to the axis B-B as shown.

In an alternative embodiment, at least one portion of the inner surface of the sidewall of the collection cup may be oriented vertically rather than being angled as shown in the figures. In such an embodiment, the channel that is configured to receive the distal portion of the stem of the handle may also be oriented vertically, which would allow for the toothbrush to be held vertically while supporting the collection cup in an upright orientation. In some embodiments, the orientation of the channel (vertical or angled) may correspond to the orientation of the inner surface of the sidewall which is adjacent to the channel to ensure that the sensor is able to obtain an accurate measurement.

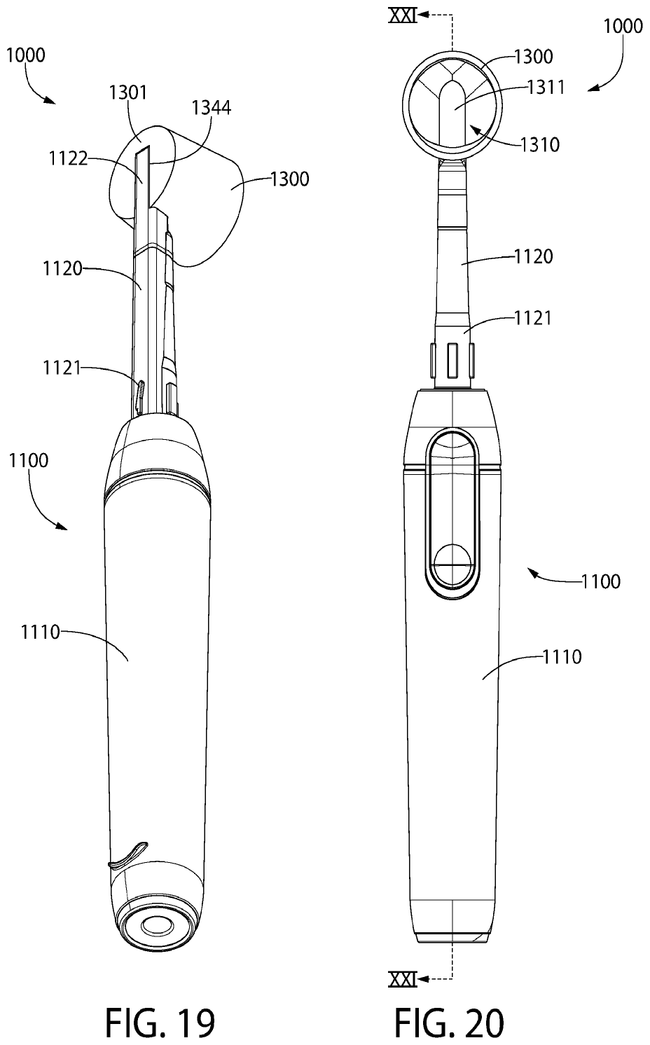

FIGS. 19-21 illustrate the oral care system 1000 whereby the collection cup 1300 is detachably coupled to the handle 1100. As noted previously, this is achieved by sliding the distal portion 1122 of the stem 1120 of the handle 1100 into the channel 1341 of the collection cup 1300. Once so coupled, the inturned portions of the walls of the channel 1341 may prevent the distal portion 1122 of the stem 1120 from being pulled through the opening 1344 of the channel 1341 which is formed into the bottom end 1301 of the collection cup 1300. Thus, when the collection cup 1300 is coupled to the handle 1100, the distal portion 1122 of the stem 1120 (where the sensor 1150 resides) is located within the channel 1341 of the collection cup 1300.

As noted above, the channel 1341 of the collection cup 1300 may be aligned with the floor 1311 of the collection cavity 1310. The floor 1311 of the collection cavity 1310 may be located directly above the channel 1341. As such, when the distal portion 1122 of the stem 1120 of the handle 1100 is located within the channel 1341 of the collection cup 1300, the sensor 1150 is aligned with the floor 1311 of the collection cavity 1310. This enables the sensor 1150 to readily take measurements of a sample of a user's saliva 1390 located in the collection cavity 1310. The sample of the user's saliva 1390 may be introduced into the collection cavity 1310 by a user directly spitting into the collection cavity 1310 or by scooping or otherwise removing (via a syringe or otherwise) the sample out of the oral cavity of the user and introducing it into the collection cavity 1310.

When the collection cup 1300 is coupled to the stem 1120, the proximal portion 1121 of the stem 1120 may be exposed. That is, the distal portion 1122 of the stem 1120 may be located within the channel 1341, but the proximal portion 1121 may not be covered by the collection cup 1300. Thus, the collection cup 1300 may be attached only to the distal portion 1122 of the stem 1120, leaving the rest of the stem 1120 uncovered and exposed. The proximal portion 1121 may not fit within the channel 1341 due to size constraints, or the distal end of the stem 1120 may abut against the end wall of the channel 1341 when the collection cup 1300 is fully coupled to the stem 1120 of the handle 1100.

In embodiments whereby the collection cup 1300a is used, the sensor 1150 will still be aligned with the collection cavity 1310a despite not being located below the floor 1311a of the collection cavity 1310a. Rather, the sensor 1150 will be located alongside of the collection cavity 1310a and positioned adjacent to the floor 1311a of the collection cavity 1310a to ensure that the sensor 1150 is still able to take measurements of a sample of saliva located on the floor 1311a of the collection cavity 1310a.

As best shown in FIG. 21, the handle 1100 may comprise a power source 1199 for powering the sensor 1150. The handle 1100 may comprise additional electronic components, including those described above with reference to FIG. 12.

In accordance with one embodiment of the present invention, a user may use the oral care system 1000 as follows. First, the user may attach the refill head 1200 to the handle 1100 and perform a toothbrushing session therewith. After completion of the toothbrushing session, the user may detach the refill head 1200 from the handle 1100. The user may then attach the collection cup 1300 to the handle 1100 in the manner described herein. Next, and prior to rinsing the user's mouth/oral cavity, the user may introduce a sample of saliva/toothpaste slurry into the collection cavity 1310 of the collection cup 1300. The user may introduce the saliva/toothpaste slurry into the collection cavity 131 before attaching the collection cup 1300 to the handle 1100 in some embodiments, although this can be done after the collection cup 1300 is attached to the handle 110 in other embodiments. Next, the sensor 1150 may be activated to emit the various lights at the various wavelengths as described herein into the collection cavity 1310. The light from the sensor 1150 may be directed towards the sample 1390 in the collection cavity 1310. The sensor 1150 will receive reflected portions of the light as described herein. Next, signals indicative of the intensities of the various reflected portions of the light may be transmitted to a processor. The processor may be located within the handle 1100 or may be part of a separate electronic device as described herein. The processor may then calculate ratios of the intensities of the different reflected lights to determine whether hemoglobin is present in the sample. The details of the operation of the sensor 1150 and process are exactly as described above and are not repeated herein the interest of brevity.

Once measurements are complete, the collection cup 1300 may be detached from the handle 1100 and rinsed. In some embodiments, the sensor 1150 may require periodic calibration. In such embodiments, an object such as paper, a calibration solution, or the like may be placed on the floor 1311 of the collection cavity 1310 while the collection cup 1300 is coupled to the handle 1100. The sensor 1150 may then be activated and calibrated with the paper or calibration solution located on the floor 1311 of the collection cavity 1310.

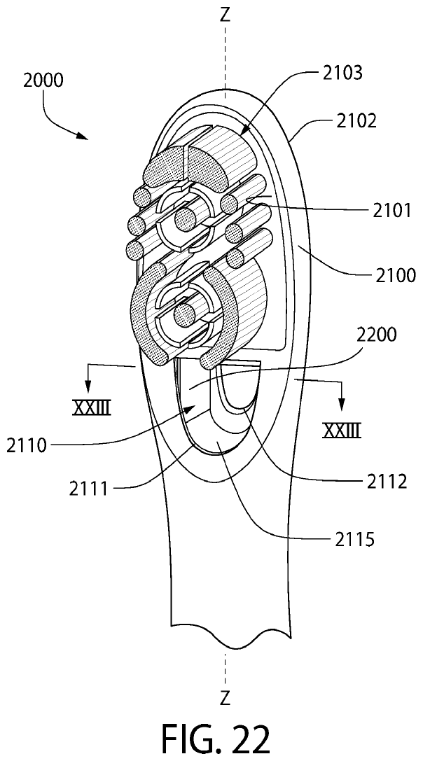

Referring to FIG. 22, an oral care implement 2000 is illustrated in accordance with another embodiment. In FIG. 22, just a portion of a head 2100 of the oral care implement 2000 is illustrated. The head 2100 may be a refill head or may be an integral part of a unitary oral care implement structure. Many of the features and concepts disclosed above are relevant to the oral care implement 2000, and thus only the differences will be described herein below. As such, it should be appreciated that the disclosure above is or may be relevant to the oral care implement 000 for all features not explicitly described with reference to the oral care implement 2000. For example, the details of the handle, the sensors, the electronic circuitry, and the like as described above may be applicable to the oral care implement 2000.

The head 2100 comprises a front surface 2101 having tooth cleaning elements 2103 extending therefrom and a rear surface 2102 opposite the front surface 2102. Furthermore, in the exemplified embodiment the head 2100 comprises a cavity 2110. In alternative embodiments, the cavity 2110 may be located along the neck or the handle of the oral care implement 2000 rather than on the head 2100, as discussed above. However, positioning the cavity 2110 on the head 2100 ensures that saliva and/or toothpaste slurry generated during toothbrushing will flow into the cavity 2110 where it can be monitored by the sensors for hemoglobin or blood detection or for any other purpose, some of which has been described herein above. In the exemplified embodiment, the cavity 2110 extends from a first opening 2111 on the front surface 2101 of the head 2100 and a second opening 2112 on the rear surface 2102 of the head 2100. The first opening 2111 may have a larger cross-sectional area than the second opening 2112 to allow a user's saliva or toothpaste slurry (saliva mixed with toothpaste) to more easily enter the cavity 2110 than exit to allow time for the sensors to perform their measurements. Thus, the cavity 2110 extends through the full thickness of the head 2100. However, the invention is not to be so limited and the cavity 2110 may be open on the front surface 2101 and closed on the rear surface 2102 such that the cavity 2110 may have a floor. Alternatively, the cavity 2110 may be open on the rear surface 2102 and closed on the front surface 2101. The cavity 2110 may be bounded by a sidewall 2115 that extends in a direction between the front and rear surfaces 2101, 2102 of the head 2100.

Furthermore, although not illustrated in FIG. 22, the oral care implement 2000 may include a closure member that is configured to close one or both of the first and second openings 2111, 2112. That is, the closure member may be alterable between a closed state wherein the first and/or second openings 2111, 2112 are closed and the cavity 2110 is inaccessible and an open state wherein the first and/or second openings 2111, 2112 are open and the cavity 2110 is accessible. The closure member may be a slide member as described above, or the closure member may be alterable between the open/closed states in other ways such as pivoting about a hinge. The closure member(s) may be alterable between the open and closed states by manual interaction by a user or automatically.

The oral care implement 2000 may comprise one or more sensors 2200 that may be used to detect blood or hemoglobin in a sample of the user's saliva, or may be used for other purposes which have been described herein.

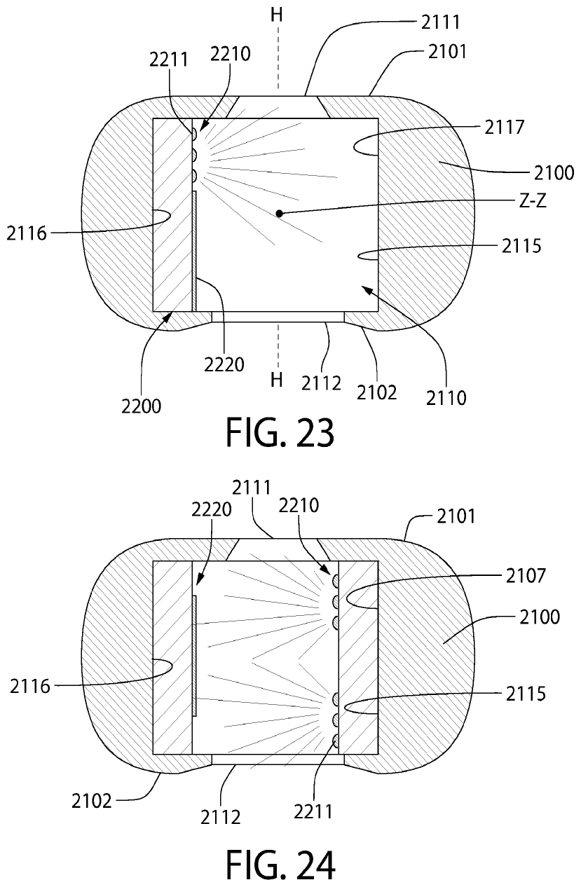

Referring to FIG. 23, one embodiment of the oral care implement 2000 will be described with specific reference to the positioning of the sensor 2200. The sensor 2200 may comprise a transmitter 2210 and a receiver 2220. The transmitter 2210 may comprise one or more light sources 2211, some details of which have been provided above and are applicable here. The details of the receiver 2220 and the manner in which it functions along with the transmitter 2210 to perform measurements are described above and those descriptions are applicable here. The main difference between the discussion above and the embodiment of the oral care implement 2000 relates to the placement and/or positioning of the sensor 2200 and the transmitter 2210 and receiver 2220 thereof.

The head 2100 of the oral care implement 2000 comprises a median plane Z-Z. The sidewall 2115 of the cavity 2110 comprises a first sidewall portion 2116 located on a first side of the median plane Z-Z and a second sidewall portion 2117 located on a second side of the median plane Z-Z. In this embodiment, the sensor 2200 is located on the first sidewall portion 2116 of the sidewall 2115 of the cavity 2110. More specifically, the transmitter 2210 and the receiver 2220 are both located on the first sidewall portion 2116 of the sidewall 2115 of the cavity 2110. Thus, the light emitted from the transmitter 2210 may emit towards a sample in the cavity 2110, bounce off the second sidewall portion 2117, and then be received by the receiver 2220 for processing.

Referring to FIG. 24, an alternative version of the head 2100 is shown. In FIG. 24, the receiver 2220 is located on the first sidewall portion 2116 and the transmitter(s) 2210 are located on the second sidewall portion 2117.

It should be noted that in both of these embodiments, the sensors 2200 are oriented so that they do not face towards either of the openings 2111, 2112 into the cavity 2110. That is, the cavity 2110 has a cavity axis H-H that extends from the opening 2111 in the front surface 2101 to the opening 2112 in the rear surface 2102 (or, where there are not two openings, the cavity axis H-H extends between the front and rear surfaces 2101, 2102 of the head 2100). The transmitter 2110 is oriented so that it transmits light (or electromagnetic radiation) in a direction that is generally transverse to the cavity axis H-H. For example, if the sensors 2200 were located on the floor of the cavity and facing upward, the sensors 2200 would potentially face towards the mouth where there are many red objects, such as the lips, cheeks, and tongue. When sensors of this type are facing towards the mouth, it is possible that the hemoglobin detection may be affected based on the detection of other red-colored objects like the lips, cheeks, and tongue. Thus, by positioning the sensors 2200 on the sidewall 2115 of the cavity 2110 as shown and described, the sensors 2200 are not impacted by red objects of the mouth and can take better hemoglobin/blood measurements. The location of the transmitters 2110 may be modified from that which is shown so that they are more centrally located to further reduce the amount of electromagnetic radiation which is able to pass through the openings 2111, 2112. Other modifications, such as selection of transmitter type and its electromagnetic radiation emittance path may be selected and modified to further enhance the function and operation.

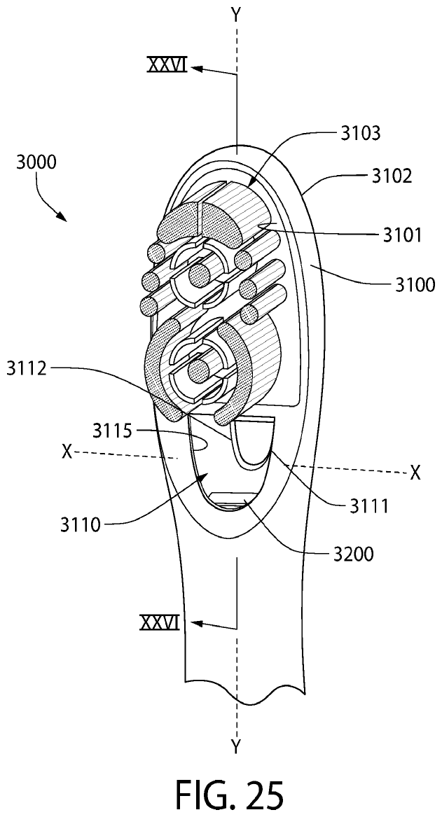

FIGS. 25-27 illustrate an embodiment similar to FIGS. 22-24, with some minor modifications. Specifically, FIG. 25 illustrates an oral care implement 3000 comprising a head 3100 having a front surface 3101 and a rear surface 3102. There are tooth cleaning elements 3103 extending from the front surface 3101. The head 3100 comprises a cavity 3110. The cavity 3110 may extend from an opening 3111 in the front surface 3101 to an opening 3112 in the rear surface 3102. Alternatively, the cavity 3110 may be open on only one of the front and rear surfaces 3101, 3102, but not the other. The cavity 3115 may be bounded or defined by a sidewall 3115. The oral care implement 3000 may comprise one or more sensors 3200 located on the sidewall 3115.

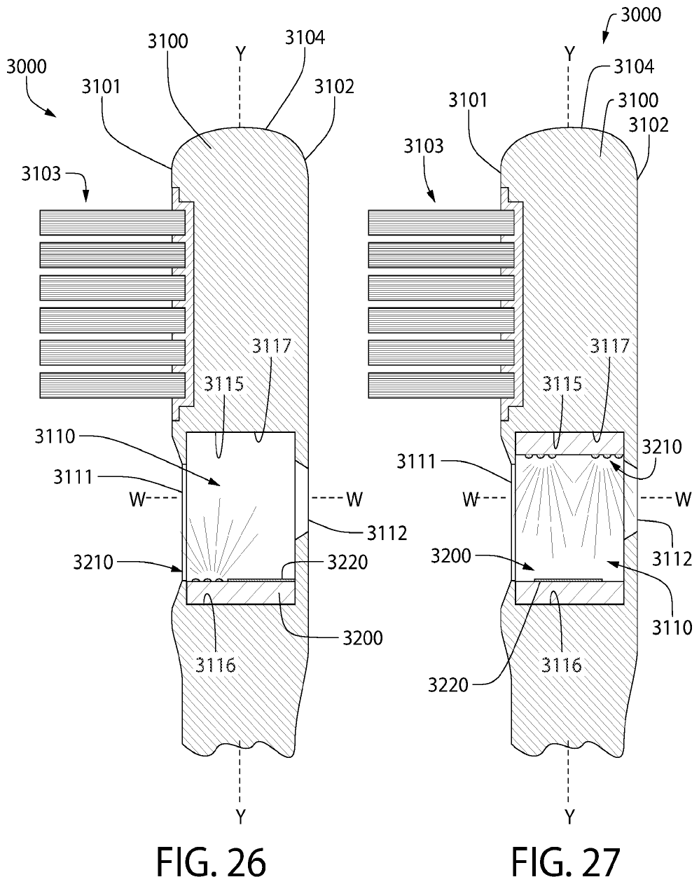

The head 3100 may have a longitudinal axis Y-Y and a transverse axis X-X, with the transverse axis X-X being perpendicular to the longitudinal axis Y-Y and passing through the cavity 3110. In this embodiment, the cavity 3110 may have a cavity axis W-W as shown in FIGS. 26 and 27. The cavity axis W-W extends from the first opening 3111 in the front surface 3101 to the second opening 3112 in the rear surface 3102. The sensor 3200 may comprise a transmitter 3210 and a receiver 3220. The sidewall 3115 may comprise a first sidewall portion 3116 located on a first side of the transverse axis X-X and a second sidewall portion 3117 located on a second side of the transverse axis X-X. The first sidewall portion 3116 may face upwardly towards a distal end 3104 of the head 3100 and the second sidewall portion 3117 may face downwardly towards a handle of the oral care implement 3000 (not illustrated).

In the embodiment shown in FIG. 26, the sensor 3200 including both the transmitter 3210 and the receiver 3220 may be located on the first sidewall portion 3116. In the embodiment shown in FIG. 27, the receiver 3220 may be located on the first sidewall portion 3116 and the transmitter 3210 may be located on the second sidewall portion 3117. In either case, the transmitter 3210 is configured to emit light in the direction of the longitudinal axis Y-Y. Stated another way, the transmitter 3210 is configured to emit light or electromagnetic radiation in a direction that is generally perpendicular to the cavity axis W-W. As such, the majority of the light is not emitted outwardly through either of the openings 3111, 3112 and into the mouth where the hemoglobin measurement may be affected by red colors of objects in the mouth such as the lips, gums, tongue, and cheeks.

In these embodiments, the user's saliva may enter into the cavity 2110, 3110 during toothbrushing. The user's saliva may be mixed with toothpaste, such that it is a toothpaste slurry (which is a mixture of toothpaste and saliva) that flows into the cavity 2110, 3110 during toothbrushing. The sensors 2200, 3200 will obtain measurements from the toothpaste slurry to determine whether there is hemoglobin/blood present in the toothpaste slurry, and if so how much. Thus, because the toothpaste slurry enters the cavity 2110, 3110 automatically during brushing, no extra steps are required by the user. Rather, the sensors 2200, 3200 will simply take the measurements as the toothpaste slurry naturally flows into the cavity 2110, 3110 during a toothbrushing session. By positioning the sensors 2200 on the sidewall 2115, 3115 of the cavity 2110, 3110 as shown in FIGS. 22-27, the red colored objects in the user's mouth will not interfere with the measurements being taken (or such interference will be reduced as compared to an embodiment where the sensor is located on the floor of the cavity facing upwards towards the front opening).

In some embodiments, the head 2100 may be formed from a translucent material, a semi-opaque material (e.g., beige colored, for example), or a light blocking material (e.g., black colored, for example). In some embodiments, the head 2100 may be formed form a black color, such as a black resin, which may be superior in terms of avoiding interference as compared to a white colored head or a translucent or semi-opaque material.

### Data Collection and Analysis

The interference from the lips and tongue was measured using prototypes that are commensurate in scope with the oral care implements 2000, 3000 of the as described herein with reference to FIGS. 22-27. The following steps were taken:

1. The toothbrush head was placed in the air to measure the baseline signal.

2. Then the lip and the tongue were placed on the entry window and exit window in the toothbrush head.

3. The absorbance was recorded and the red/green ratio was calculated in each case and is summarized in Table 1 below:

TABLE 1
Red/Green ratio recorded in air with no object (baseline),
in presence of lip and in the presence of tongue.

| | Design Described In | | |
|---|---|---|---|
| | US20220257968 (Old | New Design - | New Design - |
| | Design) | Beige Resin | Black Resin |
| Baseline | 0.702 | 0.713 | 0.723 |
| Lip | 0.725 | 0.724 | 0.27 |
| Tongue | 0.274 | 0.727 | 0.717 |

The percentage change in absorbance in the presence
of the lip and the tongue as compared to the corresponding
baseline is summarized in the Table 2 below:

| | Design Described In | | |
|---|---|---|---|
| | US20220257968 (Old | New Design - | New Design - |
| | Design) | Beige Resin | Black Resin |
| Lip | 3.36 | 1.56 | −0.31 |
| Tongue | 5.75 | 1.77 | −0.82 |

Tables 1 and 2 clearly show that the new design as described herein with specific reference to FIGS. 22-27 are improved relative to the designs shown in US Patent Publication No. 20220257968 in terms of reducing the interference from other red objects. Furthermore, the light blocking material (e.g., the use of a black resin to form the head of the toothbrush) performs better than the translucent or semi-opaque material (beige resin) in terms of reduction of interference.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the invention should be construed broadly as set forth in the appended claims.

### EXEMPLARY CLAIM SET

Exemplary claim 1. An oral care implement comprising: a body comprising a cavity configured to receive a sample of a user's saliva, the cavity comprising a first opening through which the user's saliva can be introduced into the cavity; a first closure member alterable between: (1) a closed state whereby the first closure member closes the first opening of the cavity and prevents the user's saliva from entering into or exiting the cavity through the first opening; and (2) an open state whereby the first opening provides a first passageway into the cavity through which the user's saliva may be introduced into or removed from the cavity; and at least one sensor configured to emit light into the cavity and receive reflected portions of the emitted light to obtain data useful for determining whether hemoglobin is present in the user's saliva located in the cavity.

Exemplary claim 2. The oral care implement according to exemplary claim 1 wherein the first closure member is alterable between the closed and open states by sliding relative to the body of the oral care implement.

Exemplary claim 3. The oral care implement according to exemplary claim 2 wherein the first closure member is configured to be manually altered between the closed and open states by a user or wherein the first closure member is configured to be automatically altered between the closed and open states in response to actuation of an activation element.

Exemplary claim 4. The oral care implement according to exemplary claim 2 or exemplary claim 3 wherein the first closure member comprises a door that covers the first opening when the first closure member is in the closed state and a tab protruding from the door, wherein the base comprises a slot within which the tab is configured to slide as the cover member is altered between the closed and open states.

Exemplary claim 5. The oral care implement according to any one of exemplary claims 1 to 4 wherein the cavity further comprises a second opening that forms a second passageway into the cavity through which the user's saliva may be introduced into and/or removed from the cavity, and further comprising a second closure member alterable between: (1) a closed state whereby the second closure member closes the second opening of the cavity and prevents the user's saliva from entering into or exiting the cavity through the second opening; and (2) an open state whereby the second opening forms the second passageway through which the user's saliva may be introduced into or removed from the cavity.

Exemplary claim 6. The oral care implement according to exemplary claim 5 wherein the first and second closure members are independently alterable between the closed and open states.

Exemplary claim 7. The oral care implement according to exemplary claim 5 or exemplary claim 6 wherein the body comprises a front surface and a rear surface opposite the front surface, and wherein the first opening is formed into the front surface of the body and the second opening is formed into the rear surface of the body such that the cavity is a through-hole that extends through the body from the front surface to the rear surface.

Exemplary claim 8. The oral care implement according to any one of exemplary claims 1 to 7 wherein the body comprises a handle portion, a head portion having tooth cleaning elements extending therefrom, and a neck portion located between the handle and head portions, and wherein the cavity is located along the neck portion of the body.

Exemplary claim 9. The oral care implement according to any one of exemplary claims 1 to 7 wherein the body comprises: a handle having a gripping portion and a stem portion; and a refill head that is detachably coupled to the handle, the refill head comprising a head portion and a sleeve portion defining a sleeve cavity within which the stem portion of the handle nests when the refill head is coupled to the handle, and further comprising a plurality of tooth cleaning elements coupled to and extending from the head portion of the refill head.

Exemplary claim 10. The oral care implement according exemplary claim 9 wherein the at least one sensor is located along the stem portion of the handle, the at least one sensor being adjacent to or aligned with the cavity when the refill head is coupled to the handle

Exemplary claim 11. The oral care implement according to exemplary claim 9 or exemplary claim 10 wherein the cavity is formed in the sleeve portion of the refill head, wherein the first opening is located on a front surface of the sleeve portion of the refill head, and wherein the first closure member is slidable relative to the sleeve portion of the refill head between the closed and open states.

Exemplary claim 12. The oral care implement according to exemplary claim 11 wherein the refill head comprises a second opening located on a rear surface of the sleeve portion of the refill head, and further comprising a second closure member that is slidable relative to the sleeve portion of the refill head between (1) a closed state whereby the second closure member closes the second opening of the cavity and prevents the user's saliva from entering into or exiting the cavity through the second opening; and (2) an open state whereby the second opening provides the second passageway through which the user's saliva may be introduced into or removed from the cavity.

Exemplary claim 13. The oral care implement according to any one of exemplary claims 1 to 12 wherein the at least one sensor is configured to: emit first light at a first wavelength; emit second light at a second wavelength; receive reflected portions of the first light and the second light; and generate a first signal indicative of a first intensity of the reflected portion of the first light and a second signal indicative of a second intensity of the reflected portion of the second light.

Exemplary claim 14. A blood detection system comprising: the oral care implement according to exemplary claim 13; and a processor operably coupled to the at least one sensor and configured to receive the first and second signals and calculate a ratio of the first intensity to the second intensity to determine, when the sample of the user's saliva is present in the cavity or the body of the oral care implement, whether hemoglobin is detected in the sample of the user's saliva.

Exemplary claim 15. The blood detection system according to exemplary claim 14 wherein the at least one sensor is further configured to emit third light at a third wavelength, receive reflected portions of the third light, and generate a third signal indicative of a third intensity of the reflected portion of the third light, and wherein the processor is configured to receive the third signal and calculate a ratio of the third intensity to the second intensity to determine whether hemoglobin is detected in the sample of the user's saliva.

Exemplary claim 16. The blood detection system according to exemplary claim 15 wherein the first light is one of red and infrared light, the second light is green light, and the third light is the other one of red light and infrared light.

Exemplary claim 17. The blood detection system according to any one of exemplary claims 14 to 16 wherein the oral care implement comprises the processor.

Exemplary claim 18. The blood detection system according to any one of exemplary claims 14 to 16 further comprising a portable electronic device comprising the processor, wherein the oral care implement is configured to be in electronic communication with the portable electronic device to facilitate transmission of the first and second signals from the oral care implement to the processor of the portable electronic device for processing.

Exemplary claim 19. The blood detection system according to any one of exemplary claims 14 to 18 wherein the oral care implement further comprises a visual, haptic, or auditory indicator to provide various indications to the user regarding the presence or absence of hemoglobin in the sample of the user's saliva.

Exemplary claim 20. The blood detection system according to exemplary claim 19 wherein the visual, haptic, or auditory indicator provides an indication to a user when an amount of hemoglobin detected in the sample of the user's saliva is above a threshold.

Exemplary claim 21. An oral care system comprising: a handle comprising a gripping portion and a stem portion, the handle comprising at least one sensor; a refill head comprising a sleeve portion and a head portion having tooth cleaning elements extending therefrom, the refill head configured to be detachably coupled to the handle; a collection cup comprising a collection cavity, the collection cup configured to be detachably coupled to the handle; and wherein when the collection cup is coupled to the handle and a sample of a user's saliva is located in the collection cavity, the at least one sensor is configured to obtain data useful for determining whether hemoglobin is present in the sample of the user's saliva located in the collection cavity.

Exemplary claim 22. The oral care system according to exemplary claim 21 further comprising: the handle comprising a first connection feature and a second connection feature; the refill head comprising a third connection feature, the refill head configured to be detachably coupled to the handle via engagement between the first connection feature of the handle and the third connection feature of the refill head; and the collection cup comprising a fourth connection feature, the collection cup configured to be detachably coupled to the handle via engagement between the second engagement feature of the handle and the fourth engagement feature of the collection cup.

Exemplary claim 23. The oral care system according to exemplary claim 22 wherein the stem portion of the handle comprises a proximal portion located adjacent to the gripping portion and a distal portion located furthest from the gripping portion, the proximal portion comprising the first connection feature and the distal portion comprising the second connection feature.

Exemplary claim 24. The oral care system according to exemplary claim 23 wherein when the refill head is coupled to the handle, the proximal and distal portions of the stem nest within a cavity of the refill head, and wherein the collection cup comprises a channel such that when the collection cup is coupled to the handle, the distal portion of the stem nests within the channel of the collection cup and the proximal portion of the stem remains exposed.

Exemplary claim 25. The oral care system according to exemplary claim 24 wherein the at least one sensor is located along the distal portion of the stem so that the at least one sensor is aligned with the collection cavity of the collection cup when the collection cup is coupled to the handle.

Exemplary claim 26. The oral care system according to any one of exemplary claims 21 to 25 wherein the tooth cleaning elements define a cleaning element field, the cleaning element field comprising an opening that forms a passageway from a distal end of the cleaning element field to a front surface of the head portion of the refill head, the passageway being aligned with the at least one sensor when the refill head is coupled to the handle, and wherein the at least one sensor is configured to obtain data useful for determining whether hemoglobin is present in the user's mouth during a toothbrushing session using the handle and the attached refill head.

Exemplary claim 27. The oral care system according to any one of exemplary claims 21 to 26 wherein the sensor is located along a distal portion of the stem of the handle, wherein the distal portion of the stem of the handle is aligned with the head portion of the refill head when the refill head is coupled to the handle, and wherein the distal portion of the stem is aligned with the collection cavity of the collection cup when the collection cup is coupled to the handle.

Exemplary claim 28. The oral care system according to any one of exemplary claims 21 to 27 wherein the collection cavity of the collection cup comprises a floor and a sidewall, the collection cup comprising a top end, a bottom end, and a base portion located between the floor of the collection cavity and the bottom end of the collection cup, the base portion comprising a channel that is configured to receive at least a distal portion of the stem of the handle to couple the collection cup to the handle.

Exemplary claim 29. The oral care system according to exemplary claim 28 wherein the collection cavity comprises a cavity axis that extends vertically between the top and bottom ends of the collection cup, and wherein the channel is elongated along a channel axis that is perpendicular to the cavity axis.

Exemplary claim 30. The oral care system according to exemplary claim 28 or exemplary claim 29 wherein the sidewall of the collection cup comprises an inner surface, wherein portions of the inner surface of the sidewall are angled towards a cavity axis of the collection cavity moving in a direction from the top end of the collection cup to the bottom end of the collection cup.

Exemplary claim 31. The oral care system according to any one of exemplary claims 28 to 30 wherein the floor of the collection cup comprises a two-dimensional spherocylindrical shape.

Exemplary claim 32. The oral care system according to any one of exemplary claims 28 to 31 wherein the channel of the collection cup is located directly below the floor of the collection cup, the floor and the channel being elongated along axes that are parallel to one another.

Exemplary claim 33. The oral care system according to any one of exemplary claims 28 to 32 wherein the channel terminates in an end wall, the end wall comprising at least one hole, and wherein the handle comprises at least one protrusion extending from a distal end of the stem portion, the at least one protrusion being received within the at least one hole when the collection cup is coupled to the handle.

Exemplary claim 34. The oral care system according to any one of exemplary claims 21 to 33 wherein the at least one sensor is configured to: emit first light at a first wavelength; emit second light at a second wavelength; receive reflected portions of the first light and the second light; and generate a first signal indicative of a first intensity of the reflected portion of the first light and a second signal indicative of a second intensity of the reflected portion of the second light.

Exemplary claim 35. An oral care system according to exemplary claim 34 further comprising a processor operably coupled to the at least one sensor and configured to receive the first and second signals and calculate a ratio of the first intensity to the second intensity to determine whether hemoglobin is present in the sample of the user's saliva.

Exemplary claim 36. The oral care system according to exemplary claim 35 wherein the at least one sensor is further configured to emit third light at a third wavelength, receive reflected portions of the third light, and generate a third signal indicative of a third intensity of the reflected portion of the third light, and wherein the processor is configured to receive the third signal and calculate a ratio of the third intensity to the second intensity to determine whether hemoglobin is present in the sample of the user's saliva.

Exemplary claim 37. The oral care system according to exemplary claim 36 wherein the first light is one of red and infrared light, the second light is green light, and the third light is the other one of red light and infrared light.

Exemplary claim 38. The oral care system according to any one of exemplary claims 35 to 37 wherein the handle comprises the processor.

Exemplary claim 39. The oral care system according to any one of exemplary claims 35 to 37 wherein the handle is part of an oral care implement, and further comprising a portable electronic device comprising the processor, wherein the oral care implement is configured to be in electronic communication with the portable electronic device to facilitate transmission of the first and second signals to the processor of the portable electronic device for processing.

Exemplary claim 40. The oral care system according to any one of exemplary claims 21 to 39 wherein the collection cup comprises a channel that is configured to receive at least a distal portion of the stem of the handle to couple the collection cup to the handle.

Exemplary claim 41. The oral care system according to exemplary claim 40 wherein the channel is formed into at least one of a base portion of the collection cup and a sidewall of the collection cup.

Exemplary claim 42. A method for detecting blood in an oral cavity, the method comprising: performing a toothbrushing session with a toothbrush comprising a handle and a refill head that is detachably coupled to the handle, the refill head comprising tooth cleaning elements that engage teeth and gums of an oral cavity of a user during the toothbrushing session; upon completion of the toothbrushing session, detaching the refill head from the handle: attaching a collection cup to the handle of the toothbrush; prior to rinsing the user's mouth, introducing a sample of saliva from the user's mouth into the collection cavity of the collection cup; emitting into the collection cavity, via a sensor located within the handle of the toothbrush, first light at a first wavelength and second light at a second wavelength; receiving, via the sensor of the toothbrush, reflected portions of the first light and the second light; and transmitting, from the sensor to a processor, a first signal indicative of a first intensity of the reflected portion of the first light and a second signal indicative of a second intensity of the reflected portion of the second light; and calculating, via the processor, a ratio of the first intensity to the second intensity to determine whether hemoglobin is present in the sample.

Exemplary claim 43. The method according to exemplary claim 42 wherein the saliva from the user's mouth is introduced into the collection cavity of the collection cup either prior to or after attaching the collection cup to the handle of the toothbrush.

Exemplary claim 44. The method according to exemplary claim 41 or exemplary claim 42 further comprising, during the toothbrushing session with the refill head coupled to the handle: emitting into the oral cavity, via the sensor, first light at the first wavelength and second light at the second wavelength during the toothbrushing session; receiving, via the sensor of the toothbrush, a second reflected portion of the first light and a second reflected portion of the second light during the toothbrushing session; transmitting, from the sensor to the processor, a third signal indicative of a third intensity of the second reflected portion of the first light and a fourth signal indicative of a fourth intensity of the second reflected portion of the second light; and calculating, via the processor, a ratio of the third intensity of the second reflected portion of the first light to the fourth intensity of the second reflected portion of the second light to determine whether hemoglobin is present in the oral cavity during the toothbrushing session.

Exemplary claim 45. The method according to any one of exemplary claims 42 to 44 wherein the first light is red or infrared light and the second light is green light.

Exemplary claim 46. An oral care system comprising: a handle comprising a gripping portion and a stem portion, the handle comprising at least one sensor; a collection cup comprising a top end, a bottom end, a collection cavity comprising a floor, and a channel that is distinct from the collection cavity; wherein the collection cup is configured to be detachably coupled to the handle by inserting at least a distal portion of the stem portion of the handle into the channel of the collection cup so that the at least one sensor of the handle is aligned with the collection cavity; and wherein when the collection cup is coupled to the handle and a sample of a user's saliva is located in the collection cavity, the at least one sensor is configured to obtain data useful for determining whether hemoglobin is present in the sample of the user's saliva located in the collection cavity.

Exemplary claim 47. The oral care system according to exemplary claim 46 wherein the channel comprises an end wall, the end wall comprising at least one hole, the handle comprising at least one protrusion extending from a distal end of the stem portion, the at least one protrusion being received within the at least one hole in the end wall when the collection cup is coupled to the handle.

Exemplary claim 48. The oral care system according to exemplary claim 45 or exemplary claim 46 wherein the channel is located beneath the floor of the collection cup, the channel and the floor extending along axes that are parallel to one another.

Exemplary claim 49. The oral care system according to any one of exemplary claims 45 to 47 wherein the floor of the collection cup comprises a two-dimensional spherocylindrical shape.

Exemplary claim 50. The oral care system according to any one of exemplary claims 45 to 49 wherein the collection cup comprises a base portion located between the floor of the collection cavity and the bottom end of the collection cup, the base portion comprising the channel.

Exemplary claim 51. The oral care system according to exemplary claim 50 wherein the channel is located entirely within the base portion of the collection cup.

Exemplary claim 52. The oral care system according to any one of exemplary claims 45 to 49 wherein the channel extends from an opening in the bottom end of the collection cup to an end wall, the channel being elongated along a channel axis that is parallel to a cavity axis of the collection cavity.

Exemplary claim 53. The oral care system according to any one of exemplary claims 45 to 52 wherein the collection cavity comprises an open top end having a first area and the floor of the collection cavity has a second area, a ratio of the first area to the second area being in a range of 3:1 and 5:1.

Exemplary claim 54. The oral care system according to any one of exemplary claims 45 to 53 wherein the at least one sensor is configured to: emit first light at a first wavelength; emit second light at a second wavelength; receive reflected portions of the first light and the second light; and generate a first signal indicative of a first intensity of the reflected portion of the first light and a second signal indicative of a second intensity of the reflected portion of the second light.

Exemplary claim 55. An oral care system according to exemplary claim 54 further comprising a processor operably coupled to the at least one sensor and configured to receive the first and second signals and calculate a ratio of the first intensity to the second intensity to determine whether hemoglobin is present in the sample of the user's saliva.

Exemplary claim 56. The oral care system according to exemplary claim 55 wherein the at least one sensor is further configured to emit third light at a third wavelength, receive reflected portions of the third light, and generate a third signal indicative of a third intensity of the reflected portion of the third light, and wherein the processor is configured to receive the third signal and calculate a ratio of the third intensity to the second intensity to determine whether hemoglobin is present in the sample of the user's saliva.

Exemplary claim 57. The oral care system according to exemplary claim 56 wherein the first light is one of red and infrared light, the second light is green light, and the third light is the other one of red light and infrared light.

Exemplary claim 58. The oral care system according to any one of exemplary claims 55 to 57 wherein the handle comprises the processor.

Exemplary claim 59. The oral care system according to any one of exemplary claims 55 to 57 wherein the handle is part of an oral care implement, and further comprising a portable electronic device comprising the processor, wherein the oral care implement is configured to be in electronic communication with the portable electronic device to facilitate transmission of the first and second signals to the processor of the portable electronic device for processing.

Exemplary claim 60. An oral care system comprising: a handle comprising a gripping portion and a stem portion, the handle comprising at least one sensor; a collection cup comprising a collection cavity; wherein the collection cup is configured to be detachably coupled to the handle so that the at least one sensor of the handle is adjacent to or aligned with the collection cavity; and; wherein when the collection cup is coupled to the handle and a sample of a user's saliva is located in the collection cavity, the at least one sensor is configured to obtain data useful for determining whether hemoglobin is present in the sample of the user's saliva located in the collection cavity.

1. An oral care implement comprising:
a body comprising a cavity configured to receive a sample of a user's saliva, the cavity comprising a first opening through which the user's saliva can be introduced into the cavity;
a first closure member alterable between: (1) a closed state whereby the first closure member closes the first opening of the cavity and prevents the user's saliva from entering into or exiting the cavity through the first opening; and (2) an open state whereby the first opening provides a first passageway into the cavity through which the user's saliva may be introduced into or removed from the cavity; and
at least one sensor configured to emit light into the cavity and receive reflected portions of the emitted light to obtain data useful for determining whether hemoglobin is present in the user's saliva located in the cavity.

2. The oral care implement according to claim 1 wherein the first closure member is alterable between the closed and open states by sliding relative to the body of the oral care implement.

3. The oral care implement according to claim 2 wherein the first closure member comprises a door that covers the first opening when the first closure member is in the closed state and a tab protruding from the door, wherein the base comprises a slot within which the tab is configured to slide as the cover member is altered between the closed and open states.

4. The oral care implement according to claim 1 wherein the cavity further comprises a second opening that forms a second passageway into the cavity through which the user's saliva may be introduced into and/or removed from the cavity, and further comprising a second closure member alterable between: (1) a closed state whereby the second closure member closes the second opening of the cavity and prevents the user's saliva from entering into or exiting the cavity through the second opening; and (2) an open state whereby the second opening forms the second passageway through which the user's saliva may be introduced into or removed from the cavity, wherein the first and second closure members are independently alterable between the closed and open states.

5. (canceled)

6. The oral care implement according to claim 4 wherein the body comprises a front surface and a rear surface opposite the front surface, and wherein the first opening is formed into the front surface of the body and the second opening is formed into the rear surface of the body such that the cavity is a through-hole that extends through the body from the front surface to the rear surface.

7. The oral care implement according to claim 1 wherein the body comprises a handle portion, a head portion having tooth cleaning elements extending therefrom, and a neck portion located between the handle and head portions, and wherein the cavity is located along the neck portion of the body.

8. The oral care implement according to claim 1 wherein the at least one sensor is configured to:
emit first light at a first wavelength;
emit second light at a second wavelength;
receive reflected portions of the first light and the second light; and
generate a first signal indicative of a first intensity of the reflected portion of the first light and a second signal indicative of a second intensity of the reflected portion of the second light.

9. A blood detection system comprising:
the oral care implement according to claim 8; and
a processor operably coupled to the at least one sensor and configured to receive the first and second signals and calculate a ratio of the first intensity to the second intensity to determine, when the sample of the user's saliva is present in the cavity or the body of the oral care implement, whether hemoglobin is detected in the sample of the user's saliva.

10. (canceled)

11. The blood detection system according to claim 9 wherein the oral care implement further comprises a visual, haptic, or auditory indicator to provide various indications to the user regarding the presence or absence of hemoglobin in the sample of the user's saliva.

12. An oral care system comprising:
a handle comprising a gripping portion and a stem portion, the handle comprising at least one sensor;
a refill head comprising a sleeve portion and a head portion having tooth cleaning elements extending therefrom, the refill head configured to be detachably coupled to the handle;
a collection cup comprising a collection cavity, the collection cup configured to be detachably coupled to the handle; and
wherein when the collection cup is coupled to the handle and a sample of a user's saliva is located in the collection cavity, the at least one sensor is configured to obtain data useful for determining whether hemoglobin is present in the sample of the user's saliva located in the collection cavity.

13. The oral care system according to claim 12 further comprising:
the handle comprising a first connection feature and a second connection feature;
the refill head comprising a third connection feature, the refill head configured to be detachably coupled to the handle via engagement between the first connection feature of the handle and the third connection feature of the refill head; and
the collection cup comprising a fourth connection feature, the collection cup configured to be detachably coupled to the handle via engagement between the second engagement feature of the handle and the fourth engagement feature of the collection cup.

14. The oral care system according to claim 13 wherein the stem portion of the handle comprises a proximal portion located adjacent to the gripping portion and a distal portion located furthest from the gripping portion, the proximal portion comprising the first connection feature and the distal portion comprising the second connection feature.

15. The oral care system according to claim 14 wherein when the refill head is coupled to the handle, the proximal and distal portions of the stem nest within a cavity of the refill head, and wherein the collection cup comprises a channel such that when the collection cup is coupled to the handle, the distal portion of the stem nests within the channel of the collection cup and the proximal portion of the stem remains exposed.

16. The oral care system according to claim 15 wherein the at least one sensor is located along the distal portion of the stem so that the at least one sensor is aligned with the collection cavity of the collection cup when the collection cup is coupled to the handle.

17. The oral care system according to claim 12 wherein the collection cavity of the collection cup comprises a floor and a sidewall, the collection cup comprising a top end, a bottom end, and a base portion located between the floor of the collection cavity and the bottom end of the collection cup, the base portion comprising a channel that is configured to receive at least a distal portion of the stem of the handle to couple the collection cup to the handle, wherein the collection cavity comprises a cavity axis that extends vertically between the top and bottom ends of the collection cup, and wherein the channel is elongated along a channel axis that is perpendicular to the cavity axis.

18. (canceled)

19. The oral care system according to claim 17 wherein the sidewall of the collection cup comprises an inner surface, wherein portions of the inner surface of the sidewall are angled towards a cavity axis of the collection cavity moving in a direction from the top end of the collection cup to the bottom end of the collection cup.

20. The oral care system according to claim 17 wherein the floor of the collection cup comprises a two-dimensional spherocylindrical shape.

21. The oral care system according to claim 17 wherein the channel of the collection cup is located directly below the floor of the collection cup, the floor and the channel being elongated along axes that are parallel to one another.

22. The oral care system according to claim 17 wherein the channel terminates in an end wall, the end wall comprising at least one hole, and wherein the handle comprises at least one protrusion extending from a distal end of the stem portion, the at least one protrusion being received within the at least one hole when the collection cup is coupled to the handle.

23. (canceled)

24. (canceled)

25. An oral care system comprising:
a handle comprising a gripping portion and a stem portion, the handle comprising at least one sensor;
a collection cup comprising a collection cavity;
wherein the collection cup is configured to be detachably coupled to the handle so that the at least one sensor of the handle is adjacent to or aligned with the collection cavity; and
wherein when the collection cup is coupled to the handle and a sample of a user's saliva is located in the collection cavity, the at least one sensor is configured to obtain data useful for determining whether hemoglobin is present in the sample of the user's saliva located in the collection cavity.
